(19) United States
(12) Patent Application Publication
(10) Pub. No.: US 2022/0218612 A1
(43) Pub. Date: Jul. 14, 2022

(54) IMPROVED PROCESS OF PREPARING MRNA-LOADED LIPID NANOPARTICLES

(71) Applicant: Translate Bio, Inc., Lexington, MA (US)

(72) Inventors: Shrirang Karve, Lexington, MA (US); Frank DeRosa, Lexington, MA (US); Michael Hearlein, Lexington, MA (US); Ashish Sarode, Lexington, MA (US); Zarna Patel, Lexington, MA (US); Rebecca L. Ball, Lexington, MA (US); Natalia Vargas Montoya, Lexington, MA (US); Priyal Patel, Lexington, MA (US); Asad Khanmohammed, Lexington, MA (US)

(21) Appl. No.: 17/611,020

(22) PCT Filed: May 14, 2020
(86) PCT No.: PCT/US2020/032943

§ 371 Date: Nov. 12, 2021

Related U.S. Application Data
(60) Provisional application No. 62/847,837, filed on May 14, 2019.

(51) Int. Cl. A61K 9/127; A61K 9/00; A61K 31/7088; A61K 9/16

(52) U.S. Cl. CPC A61K 9/1271; A61K 9/0019; A61K 9/1694; A61K 9/0073; A61K 9/1664; A61K 31/7088

(57) ABSTRACT

The present invention provides an improved process for lipid nanoparticle formulation and mRNA encapsulation. In some embodiments, the present invention provides a process for enhanced encapsulation of messenger RNA (mRNA) in lipid nanoparticles comprising a step of heating the mRNA-encapsulated lipid nanoparticles in a drug product formulation solution.

### CROSS-REFERENCE TO RELATED APPLICATIONS

This application claims priority to U.S. provisional patent application Ser. No. 62/847,837, filed May 14, 2019, which is hereby incorporated by reference in their entirety for all purposes.

### BACKGROUND

Messenger RNA therapy (MRT) is becoming an increasingly important approach for the treatment of a variety of diseases. MRT involves administration of messenger RNA (mRNA) to a patient in need of the therapy for production of the protein encoded by the mRNA within the patient's body. Lipid nanoparticles are commonly used to encapsulate mRNA for efficient in vivo delivery of mRNA.

To improve lipid nanoparticle delivery, much effort has focused on identifying novel lipids or particular lipid compositions that can affect intracellular delivery and/or expression of mRNA, e.g., in various types of mammalian tissue, organs and/or cells (e.g., mammalian liver cells). However, these existing approaches are costly, time consuming and unpredictable.

### SUMMARY OF INVENTION

The present invention provides, among other things, further improved processes for preparing mRNA-loaded lipid nanoparticles (mRNA-LNPs). The invention is based on the surprising discovery that following a process of encapsulating messenger RNA (mRNA) in LNPs comprising mixing one or more lipids in a lipid solution with one or more mRNAs in an mRNA solution to form mRNA encapsulated within LNPs (mRNA-LNPs) in a LNP formation solution (e.g., Process A as further described below), the further steps of exchanging the LNP formation solution for a drug product formulation solution and heating the mRNA-LNPs in the drug product formulation solution provide an unexpected benefit of significantly increasing the encapsulation efficiency of the mRNA-LNPs, i.e., the amount or percent of mRNA encapsulated within the LNPs (i.e., encapsulation rate or efficiency). The present invention is particularly useful for manufacturing mRNA-LNPs to have a higher encapsulation rate or efficiency as compared to conventional approaches.

As compared to conventional approaches, the inventive process described herein provides higher encapsulation efficiency and accordingly may provide higher potency and better efficacy of lipid nanoparticle delivered mRNA, thereby shifting the therapeutic index in a positive direction and providing additional advantages, such as lower cost, better patient compliance, and more patient friendly dosing regimens. mRNA-loaded lipid nanoparticle formulations provided by the present invention may be successfully delivered in vivo for more potent and efficacious protein expression via different routes of administration such as intravenous, intramuscular, intra-articular, intrathecal, inhalation (respiratory), subcutaneous, intravitreal, and ophthalmic.

This inventive process can be performed using a pump system and is therefore scalable, allowing for improved particle formation/formulation in amounts sufficient for, e.g., performance of clinical trials and/or commercial sale. Various pump systems may be used to practice the present invention including, but not limited to, pulse-less flow pumps, gear pumps, peristaltic pumps, and centrifugal pumps.

This inventive process results in superior encapsulation efficiency and homogeneous particle sizes.

Thus, in one aspect, the present invention provides a process of encapsulating messenger RNA (mRNA) in lipid nanoparticles (LNPs) comprising the steps of (a) mixing one or more lipids in a lipid solution with one or more mRNAs in an mRNA solution to form mRNA encapsulated within the LNPs (mRNA-LNPs) in a LNP formation solution; (b) exchanging the LNP formation solution for a drug product formulation solution to provide mRNA-LNP in a drug product formulation solution; and (c) heating the mRNA-LNP in the drug product formulation solution, wherein the encapsulation efficiency of the mRNA-LNPs resulting from step (c) is greater than the encapsulation efficiency of the mRNA-LNPs resulting from step (b).

In some embodiments, in step (c) the drug product formulation solution is heated by applying heat from a heat source to the solution.

In some embodiments, in step (c) the drug product formulation solution is heated by applying heat from a heat source to the solution and the solution is maintained at a temperature greater than ambient temperature for 5 seconds or more, 10 seconds or more, 20 seconds or more, 30 seconds or more, 40 seconds or more, 50 seconds or more, 1 minute or more, 2 minutes or more, 3 minutes or more 4 minute or more, 5 minutes or more, 10 minutes or more, 15 minutes or more, 20 minutes or more, 25 minutes or more, 30 minutes or more, 35 minutes or more, 40 minutes or more, 45 minutes or more, 50 minutes or more, 60 minutes or more, 70 minutes or more, 80 minutes or more, 90 minutes or more, 100 minutes or more or 120 minutes or more. In some embodiments, in step (c) the drug product formulation solution is heated by applying heat from a heat source to the solution and the solution is maintained at a temperature greater than ambient temperature for 120 minutes or less, 100 minutes or less, 90 minutes or less, 60 minutes or less, 45 minutes or less, 30 minutes or less, 25 minutes or less, 20 minutes or less, 15 minutes or less, 10 minutes or less, 5 minutes or less, 4 minutes or less, 3 minutes or less, 2 minutes or less, 1 minute or less, 50 seconds or less, 40 seconds or less, 30 seconds or less, 20 seconds or less, 10 seconds or less or 5 seconds or less. In some embodiments, in step (c) the drug product formulation solution is heated by applying heat from a heat source to the solution and the solution is maintained at a temperature greater than ambient temperature for between 10 and 20 minutes. In some embodiments, in step (c) the drug product formulation solution is heated by applying heat from a heat source to the solution and the solution is maintained at a temperature greater than ambient temperature for between 20 and 90 minutes. In some embodiments, in step (c) the drug product formulation solution is heated by applying heat from a heat source to the solution and the solution is maintained at a temperature greater than ambient temperature for between 30 and 60 minutes. In some embodiments, in step (c) the drug product formulation solution is heated by applying heat from a heat source to the solution and the solution is maintained at a temperature greater than ambient temperature for about 15 minutes. In some embodiments, the temperature to which the drug product formulation is heated (or at which the drug product formulation solution is maintained) is or is greater than about 30° C., 37° C., 40° C., 45° C., 50° C., 55° C., 60° C., 65° C., or 70° C. In some embodiments, the temperature to which the drug product formulation solution is heated ranges from about 25-70° C., about 30-70° C., about 35-70° C., about 40-70° C., about 45-70° C., about 50-70° C., or about 60-70° C. In some embodiments, the temperature greater than ambient temperature to which the drug product formulation solution is heated is about 65° C.

In some embodiments, in step (a) the lipid nanoparticles are formed by mixing lipids dissolved in the lipid solution comprising ethanol with mRNA dissolved in an aqueous mRNA solution. In some embodiments, in step (a) the one or more lipids include one or more cationic lipids, one or more helper lipids, and one or more PEG-modified lipids (also referred to as PEG lipids). In some embodiments, the lipids also contain one or more cholesterol lipids. The mRNA-LNPs are formed by the mixing of the lipid solution and the mRNA solution. Accordingly, in some embodiments, the LNPs comprise one or more cationic lipids, one or more helper lipids, and one or more PEG lipids. In some embodiments, the LNPs also contain one or more cholesterol lipids.

In some embodiments, the one or more cationic lipids are selected from the group consisting of cKK-E12, OF-02, C12-200, MC3, DLinDMA, DLinkC2DMA, ICE (Imidazol-based), HGT5000, HGT5001, HGT4003, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, 3-(4-(bis(2-hydroxydodecyl)amino)butyl)-6-(4-((2-hydroxydodecyl)(2-hydroxyundecyl)amino)butyl)-1,4-dioxane-2,5-dione (Target 23), 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione (Target 24), N1GL, N2GL, V1GL and combinations thereof.

In some embodiments, the one or more cationic lipids are amino lipids. Amino lipids suitable for use in the invention include those described in WO2017180917, which is hereby incorporated by reference. Exemplary aminolipids in WO2017180917 include those described at paragraph [0744] such as DLin-MC3-DMA (MC3), (13Z,16Z)—N,N-dimethyl-3-nonyldocosa-13,16-dien-1-amine (L608), and Compound 18. Other amino lipids include Compound 2, Compound 23, Compound 27, Compound 10, and Compound 20. Further amino lipids suitable for use in the invention include those described in WO2017112865, which is hereby incorporated by reference. Exemplary amino lipids in WO2017112865 include a compound according to one of formulae (I), (Ia1)-(Ia6), (1b), (II), (I1a), (III), (I1ia), (IV), (17-1), (19-1), (19-11), and (20-1), and compounds of paragraphs [00185], [00201], [0276]. In some embodiments, cationic lipids suitable for use in the invention include those described in WO2016118725, which is hereby incorporated by reference. Exemplary cationic lipids in WO2016118725 include those such as KL22 and KL25. In some embodiments, cationic lipids suitable for use in the invention include those described in WO2016118724, which is hereby incorporated by reference. Exemplary cationic lipids in WO2016118725 include those such as KL10, 1,2-dilinoleyloxy-N,N-dimethylaminopropane (DLin-DMA), and KL25.

In some embodiments, the one or more non-cationic lipids are selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)).

In some embodiments, the one or more PEG-modified lipids comprise a poly(ethylene) glycol chain of up to 5 kDa in length covalently attached to a lipid with alkyl chain(s) of C₆-C₂₀ length.

In some embodiments, following step (a) the mRNA-LNPs are purified by a Tangential Flow Filtration (TFF) process. In some embodiments, greater than about 50%, 55%, 60%, 65%, 70%, 75%, 80%, 85%, 90%, 95%, 96%, 97%, 98%, or 99% of the purified mRNA-LNPs have a size less than about 150 nm (e.g., less than about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, or about 50 nm). In some embodiments, substantially all of the purified mRNA-LNPs have a size less than 150 nm (e.g., less than about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, or about 50 nm). In some embodiments, greater than about 70%, 75%, 80%, 85%, 90%, 95%, 96%, 97%, 98%, 99% of the purified mRNA-LNPs have a size ranging from 50-150 nm. In some embodiments, substantially all of the purified mRNA-LNPs have a size ranging from 50-150 nm. In some embodiments, greater than about 70%, 75%, 80%, 85%, 90%, 95%, 96%, 97%, 98%, 99% of the purified mRNA-LNPs have a size ranging from 80-150 nm. In some embodiments, substantially all of the purified nanoparticles have a size ranging from 80-150 nm.

In some embodiments, a process according to the present invention results in an encapsulation efficiency following step (c) that is improved by at least 5% or more over the encapsulation efficiency following step (b). In some embodiments, a process according to the present invention results in an encapsulation efficiency following step (c) that is improved by at least 10% or more over the encapsulation efficiency following step (b). In some embodiments, a process according to the present invention results in an encapsulation efficiency following step (c) that is improved by at least 15% or more over the encapsulation efficiency following step (b). In some embodiments, a process according to the present invention results in an encapsulation efficiency following step (c) that is improved by at least 20% or more over the encapsulation efficiency following step (b). In some embodiments, a process according to the present invention results in an encapsulation efficiency following step (c) that is improved by at least 25% or more over the encapsulation efficiency following step (b).

In some embodiments, a process according to the present invention improves the encapsulation amount by 5% encapsulation or more from the encapsulation following step (b) to the encapsulation following step (c). In some embodiments, a process according to the present invention improves the encapsulation amount by 10% encapsulation or more from the encapsulation following step (b) to the encapsulation following step (c). In some embodiments, a process according to the present invention improves the encapsulation amount by 15% encapsulation or more from the encapsulation following step (b) to the encapsulation following step (c). In some embodiments, a process according to the present invention improves the encapsulation amount by 20% encapsulation or more from the encapsulation following step (b) to the encapsulation following step (c). In some embodiments, a process according to the present invention improves the encapsulation amount by 25% encapsulation or more from the encapsulation following step (b) to the encapsulation following step (c).

In some embodiments, a process according to the present invention results in greater than about 60%, 65%, 70%, 75%, 80%, 85%, 90%, 95%, 96%, 97%, 98%, or 99% recovery of mRNA following step (c).

In some embodiments, a process according to the present invention results in an encapsulation rate following step (c) of greater than about 90%, 95%, 96%, 97%, 98%, or 99%. In some embodiments, a process according to the present invention results in greater than about 60%, 65%, 70%, 75%, 80%, 85%, 90%, 95%, 96%, 97%, 98%, or 99% recovery of mRNA following step (c).

In some embodiments, the lipid solution and the mRNA solution are mixed using a pump system. In some embodiments, the pump system comprises a pulse-less flow pump. In some embodiments, the pump system is a gear pump. In some embodiments, a suitable pump is a peristaltic pump. In some embodiments, a suitable pump is a centrifugal pump. In some embodiments, the process using a pump system is performed at large scale. For example, in some embodiments, the process includes using pumps as described herein to mix a solution of at least about 1 mg, 5 mg, 10 mg, 50 mg, 100 mg, 500 mg, or 1000 mg of mRNA with a lipid solution comprising one or more cationic lipids, one or more helper lipids and one or more PEG-modified lipids. In some embodiments, the process of mixing the lipid solution and the mRNA solution provides a composition according to the present invention that contains at least about 1 mg, 5 mg, 10 mg, 50 mg, 100 mg, 500 mg, or 1000 mg of encapsulated mRNA following step (c).

In some embodiments, the lipid solution is mixed at a flow rate ranging from about 25-75 ml/minute, about 75-200 ml/minute, about 200-350 ml/minute, about 350-500 ml/minute, about 500-650 ml/minute, about 650-850 ml/minute, or about 850-1000 ml/minute. In some embodiments, the lipid solution is mixed at a flow rate of about 50 ml/minute, about 100 ml/minute, about 150 ml/minute, about 200 ml/minute, about 250 ml/minute, about 300 ml/minute, about 350 ml/minute, about 400 ml/minute, about 450 ml/minute, about 500 ml/minute, about 550 ml/minute, about 600 ml/minute, about 650 ml/minute, about 700 ml/minute, about 750 ml/minute, about 800 ml/minute, about 850 ml/minute, about 900 ml/minute, about 950 ml/minute, or about 1000 mL/minute.

In some embodiments, the mRNA solution is mixed at a flow rate ranging from about 25-75 ml/minute, about 75-200 ml/minute, about 200-350 ml/minute, about 350-500 ml/minute, about 500-650 ml/minute, about 650-850 ml/minute, or about 850-1000 ml/minute. In some embodiments, the mRNA solution is mixed at a flow rate of about 50 ml/minute, about 100 ml/minute, about 150 ml/minute, about 200 ml/minute, about 250 mi/minute, about 300 ml/minute, about 350 ml/minute, about 400 ml/minute, about 450 ml/minute, about 500 ml/minute, about 550 ml/minute, about 600 ml/minute, about 650 ml/minute, about 700 ml/minute, about 750 ml/minute, about 800 ml/minute, about 850 ml/minute, about 900 ml/minute, about 950 mi/minute, or about 1000 ml/minute.

In some embodiments, the lipid solution includes a non-aqueous solvent such as an organic solvent. In some embodiments, the lipid solution includes an alcohol. In some embodiments, the lipid solution includes ethanol. In some embodiments, a process according to the present invention includes a step of first dissolving the one or lipids in the lipid solution. In some embodiments, a process according to the present invention includes a step of first dissolving the one or lipids in the lipid solution comprising ethanol.

In some embodiments, the mRNA solution is an aqueous solution. In some embodiments, the mRNA solution comprises citrate. In some embodiments, the mRNA solution is a citrate buffer. In some embodiments, a process according to the present invention includes a step of first dissolving the mRNA in the aqueous solution. In some embodiments, a process according to the present invention includes a step of first dissolving the mRNA in the aqueous solution comprising citrate.

In some embodiments, a process according to the present invention includes a step of mixing a lipid solution comprising lipids in ethanol with a mRNA buffer comprising mRNA dissolved in citrate buffer. In some embodiments, the LNP formation solution comprises ethanol and citrate.

In some embodiments, a process according to the present invention includes a step of first generating an mRNA solution by mixing a citrate buffer with an mRNA stock solution. In certain embodiments, a suitable citrate buffer contains about 10 mM citrate, about 150 mM NaCl, pH of about 4.5. In some embodiments, a suitable mRNA stock solution contains the mRNA at a concentration at or greater than about 1 mg/ml, about 10 mg/ml, about 50 mg/ml, or about 100 mg/ml.

In some embodiments, the citrate buffer is mixed at a flow rate ranging between about 100-300 ml/minute, 300-600 ml/minute, 600-1200 mL/minute, 1200-2400 ml/minute, 2400-3600 ml/minute, 3600-4800 ml/minute, or 4800-6000 ml/minute. In some embodiments, the citrate buffer is mixed at a flow rate of about 220 ml/minute, about 600 ml/minute, about 1200 ml/minute, about 2400 ml/minute, about 3600 ml/minute, about 4800 ml/minute, or about 6000 mi/minute.

In some embodiments, the mRNA stock solution is mixed at a flow rate ranging between about 10-30 ml/minute, about 30-60 ml/minute, about 60-120 ml/minute, about 120-240 ml/minute, about 240-360 ml/minute, about 360-480 ml/minute, or about 480-600 ml/minute. In some embodiments, the mRNA stock solution is mixed at a flow rate of about 20 ml/minute, about 40 ml/minute, about 60 ml/minute, about 80 ml/minute, about 100 mi/minute, about 200 ml/minute, about 300 mi/minute, about 400 ml/minute, about 500 mi/minute, or about 600 ml/minute.

In some embodiments, in step (b) the drug product formulation solution is an aqueous solution comprising pharmaceutically acceptable excipients, including, but not limited to, a cryoprotectant. In some embodiments, in step (b) the drug product formulation solution is an aqueous solution comprising pharmaceutically acceptable excipients, including, but not limited to, a sugar. In some embodiments, in step (b) the drug product formulation solution is an aqueous solution comprising pharmaceutically acceptable excipients, including, but not limited to, one or more of trehalose, sucrose, mannose, lactose, and mannitol. In some embodiments, in step (b) the drug product formulation solution comprises trehalose. In some embodiments, in step (b) the drug product formulation solution comprises sucrose. In some embodiments, in step (b) the drug product formulation solution comprises mannose. In some embodiments, in step (b) the drug product formulation solution comprises lactose. In some embodiments, in step (b) the drug product formulation solution comprises mannitol. In some embodiments, in step (b) the drug product formulation solution is an aqueous solution comprising 5% to 20% weight to volume of a sugar, such as of trehalose, sucrose, mannose, lactose, and mannitol. In some embodiments, in step (b) the drug product formulation solution is an aqueous solution comprising 5% to 20% weight to volume of trehalose. In some embodiments, in step (b) the drug product formulation solution is an aqueous solution comprising 5% to 20% weight to volume of sucrose. In some embodiments, in step (b) the drug product formulation solution is an aqueous solution comprising 5% to 20% weight to volume of mannose. In some embodiments, in step (b) the drug product formulation solution is an aqueous solution comprising 5% to 20% weight to volume of lactose. In some embodiments, in step (b) the drug product formulation solution is an aqueous solution comprising 5% to 20% weight to volume of mannitol. In some embodiments, in step (b) the drug product formulation solution is an aqueous solution comprising about 10% weight to volume of a sugar, such as of trehalose, sucrose, mannose, lactose, and mannitol. In some embodiments, in step (b) the drug product formulation solution is an aqueous solution comprising about 10% weight to volume of trehalose. In some embodiments, in step (b) the drug product formulation solution is an aqueous solution comprising about 10% weight to volume of sucrose. In some embodiments, in step (b) the drug product formulation solution is an aqueous solution comprising about 10% weight to volume of mannose. In some embodiments, in step (b) the drug product formulation solution is an aqueous solution comprising about 10% weight to volume of lactose. In some embodiments, in step (b) the drug product formulation solution is an aqueous solution comprising about 10% weight to volume of mannitol.

In some embodiments, one or both of a non-aqueous solvent, such as ethanol, and citrate are absent (i.e., below detectable levels) from the drug product formulation solution. In some embodiments, citrate is absent (i.e., below detectable levels) from the drug product formulation solution. In some embodiments, ethanol is absent (i.e., below detectable levels) from the drug product formulation solution. In some embodiments, the drug product formulation solution comprises ethanol, but not citrate (i.e., below detectable levels). In some embodiments, the drug product formulation solution comprises citrate, but not ethanol (i.e., below detectable levels). In some embodiments, the drug product formulation solution includes only residual citrate. In some embodiments, the drug product formulation solution includes only residual non-aqueous solvent, such as ethanol. In some embodiments, the drug product formulation solution contains less than about 10 mM (e.g., less than about 9 mM, about 8 mM, about 7 mM, about 6 mM, about 5 mM, about 4 mM, about 3 mM, about 2 mM, or about 1 mM) of citrate. In some embodiments, the drug product formulation solution contains less than about 25% (e.g., less than about 20%, about 15%, about 10%, about 5%, about 4%, about 3%, about 2%, or about 1%) of non-aqueous solvents, such as ethanol. In some embodiments, the drug product formulation solution does not require any further downstream processing (e.g., buffer exchange and/or further purification steps) prior to lyophilization. In some embodiments, the drug product formulation solution does not require any further downstream processing (e.g., buffer exchange and/or further purification steps) prior to administration to a subject.

In some embodiments, the drug product formulation solution has a pH between pH 4.5 and pH 7.5. In some embodiments, the drug product formulation solution has a pH between pH 5.0 and pH 7.0. In some embodiments, the drug product formulation solution has a pH between pH 5.5 and pH 7.0. In some embodiments, the drug product formulation solution has a pH above pH 4.5. In some embodiments, the drug product formulation solution has a pH above pH 5.0. In some embodiments, the drug product formulation solution has a pH above pH 5.5. In some embodiments, the drug product formulation solution has a pH above pH 6.0. In some embodiments, the drug product formulation solution has a pH above pH 6.5.

In some embodiments, the present invention is used to encapsulate mRNA containing one or more modified nucleotides. In some embodiments, one or more nucleotides is modified to a pseudouridine. In some embodiments, one or more nucleotides is modified to a 5-methylcytidine. In some embodiments, the present invention is used to encapsulate mRNA that is unmodified.

In yet another aspect, the present invention provides a method of delivering mRNA for in vivo protein production comprising administering into a subject a composition of lipid nanoparticles encapsulating mRNA generated by the process described herein, wherein the mRNA encodes one or more protein(s) or peptide(s) of interest.

In this application, the use of “or” means “and/or” unless stated otherwise. As used in this disclosure, the term “comprise” and variations of the term, such as “comprising” and “comprises,” are not intended to exclude other additives, components, integers or steps. As used in this application, the terms “about” and “approximately” are used as equivalents. Both terms are meant to cover any normal fluctuations appreciated by one of ordinary skill in the relevant art.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only and not for limitation.

FIG. 1 shows a schematic of an conventional LNP-mRNA encapsulation process (Process A) that involves mixing mRNA dissolved in an aqueous mRNA solution with lipids dissolved in a lipid solution using a pump system to generate mRNA-LNPs in a LNP formation solution and then exchanging the LNP formation solution for a drug product formulation solution.

FIG. 2 shows a schematic of an exemplary LNP-mRNA encapsulation process of the present invention that involves mixing mRNA dissolved in an aqueous mRNA solution with lipids dissolved in a lipid solution using a pump system to generate mRNA-LNPs in a LNP formation solution, then exchanging the LNP formation solution for a drug product formulation solution, and then heating the drug product formulation solution to increase encapsulation of mRNA in the LNPs.

FIG. 3 shows the difference in encapsulation before and after a final step of heating mRNA-LNPs in drug product formulation solution, for twelve different mRNA-LNPs tested.

FIG. 4 shows the difference in encapsulation before and after a final step of heating mRNA-LNPs in drug product formulation solution, for thirteen different mRNA-LNPs tested.

FIG. 5 shows exemplary graph of protein expression after pulmonary administration of mRNA encapsulated in lipid nanoparticles prepared by Process A after a heating step.

### DEFINITIONS

In order for the present invention to be more readily understood, certain terms are first defined below. Additional definitions for the following terms and other terms are set forth throughout the specification.

Alkyl: As used herein, “alkyl” refers to a radical of a straight-chain or branched saturated hydrocarbon group having from 1 to 20 carbon atoms (“C₁₋₂₀ alkyl”). In some embodiments, an alkyl group has 1 to 3 carbon atoms (“C₁₋₃ alkyl”). Examples of C₁₋₃ alkyl groups include methyl (C₁), ethyl (C₂), n-propyl (C₃), and isopropyl (C₃). In some embodiments, an alkyl group has 8 to 12 carbon atoms (“C₈₋₁₂ alkyl”). Examples of C₈₋₁₂ alkyl groups include, without limitation, n-octyl (C₈), n-nonyl (C₉), n-decyl (C₁₀), n-undecyl (C₁₁), n-dodecyl (C₁₂) and the like. The prefix “n-” (normal) refers to unbranched alkyl groups. For example, n-C₈ alkyl refers to —(CH₂)₇CH₃, n-C₁₀ alkyl refers to —(CH₂))CH₃, etc.

Amino acid: As used herein, term “amino acid,” in its broadest sense, refers to any compound and/or substance that can be incorporated into a polypeptide chain. In some embodiments, an amino acid has the general structure H₂N—C(HXR)—COOH. In some embodiments, an amino acid is a naturally occurring amino acid. In some embodiments, an amino acid is a synthetic amino acid; in some embodiments, an amino acid is a d-amino acid; in some embodiments, an amino acid is an l-amino acid. “Standard amino acid” refers to any of the standard l-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

Efficacy: As used herein, the term “efficacy,” or grammatical equivalents, refers to an improvement of a biologically relevant endpoint, as related to delivery of mRNA that encodes a relevant protein or peptide. In some embodiments, the biological endpoint is protecting against an ammonium chloride challenge at certain timepoints after administration.

Encapsulation: As used herein, the term “encapsulation,” or grammatical equivalent, refers to the process of confining an individual mRNA molecule within a nanoparticle.

Expression: As used herein, “expression” of a mRNA refers to translation of an mRNA into a peptide (e.g., an antigen), polypeptide, or protein (e.g., an enzyme) and also can include, as indicated by context, the post-translational modification of the peptide, polypeptide or fully assembled protein (e.g., enzyme). In this application, the terms “expression” and “production,” and grammatical equivalent, are used inter-changeably.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control sample or subject (or multiple control samples or subjects) in the absence of the treatment described herein. A “control sample” is a sample subjected to the same conditions as a test sample, except for the test article. A “control subject” is a subject afflicted with the same form of disease as the subject being treated, who is about the same age as the subject being treated.

Impurities: As used herein, the term “impurities” refers to substances inside a confined amount of liquid, gas, or solid, which differ from the chemical composition of the target material or compound. Impurities are also referred to as contaminants.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 900%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Local distribution or delivery: As used herein, the terms “local distribution,” “local delivery,” or grammatical equivalent, refer to tissue specific delivery or distribution. Typically, local distribution or delivery requires a peptide or protein (e.g., enzyme) encoded by mRNAs be translated and expressed intracellularly or with limited secretion that avoids entering the patient's circulation system.

messenger RNA (mRNA): As used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Potency: As used herein, the term “potency,” or grammatical equivalents, refers to expression of protein(s) or peptide(s) that the mRNA encodes and/or the resulting biological effect.

Salt: As used herein the term “salt” refers to an ionic compound that does or may result from a neutralization reaction between an acid and a base.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Yield: As used herein, the term “yield” refers to the percentage of mRNA recovered after encapsulation as compared to the total mRNA as starting material. In some embodiments, the term “recovery” is used interchangeably with the term “yield”.

### DETAILED DESCRIPTION

The present invention provides an improved process for lipid nanoparticle formulation and mRNA encapsulation. In some embodiments, the present invention provides a process of encapsulating messenger RNA (mRNA) in lipid nanoparticles comprising the steps of (a) mixing one or more lipids in a lipid solution with one or more mRNAs in an mRNA solution to form mRNA encapsulated within the LNPs (mRNA-LNPs) in a LNP formation solution; (b) exchanging the LNP formation solution for a drug product formulation solution to provide mRNA-LNP in a drug product formulation solution; and (c) heating the mRNA-LNP in the drug product formulation solution. It was surprisingly found that inclusion of step (c) in this process provides for significantly higher encapsulation of the mRNA-LNPs as compared to the encapsulation of the same mRNA-LNPs following step (b).

In some embodiments, the novel formulation process results in an mRNA formulation with higher potency (peptide or protein expression) and higher efficacy (improvement of a biologically relevant endpoint) both in vitro and in vivo with potentially better tolerability as compared to the same mRNA formulation prepared without the additional step of heating the mRNA-LNP in the drug product formulation solution (step (c)). The higher potency and/or efficacy of such a formulation can provide for lower and/or less frequent dosing of the drug product. In some embodiments, the invention features an improved lipid formulation comprising a cationic lipid, a helper lipid and a PEG-modified lipid.

In some embodiments, the resultant encapsulation for an mRNA-LNP following step (c) is increased by 10% or more relative to the encapsulation efficiency for the same mRNA-LNP following step (b). In some embodiments, the resultant encapsulation percent for an mRNA-LNP following step (c) is increased by five percentage points or more over the encapsulation percent for the same mRNA-LNP following step (b). For the delivery of nucleic acids, achieving high encapsulation efficiencies is critical to attain protection of the drug substance and reduce loss of activity in vivo.

Various aspects of the invention are described in detail in the following sections. The use of sections is not meant to limit the invention. Each section can apply to any aspect of the invention.

Messenger RNA (mRNA)

The present invention may be used to encapsulate any mRNA. mRNA is typically thought of as the type of RNA that carries information from DNA to the ribosome. Typically, in eukaryotic organisms, mRNA processing comprises the addition of a “cap” on the 5′ end, and a “tail” on the 3′ end. A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. The additional of a tail is typically a polyadenylation event whereby a polyadenylyl moiety is added to the 3′ end of the mRNA molecule. The presence of this “tail” serves to protect the mRNA from exonuclease degradation. Messenger RNA is translated by the ribosomes into a series of amino acids that make up a protein.

mRNAs may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, in vitro synthesized mRNA may be purified before formulation and encapsulation to remove undesirable impurities including various enzymes and other reagents used during mRNA synthesis.

The present invention may be used to formulate and encapsulate mRNAs of a variety of lengths. In some embodiments, the present invention may be used to formulate and encapsulate in vitro synthesized mRNA of or greater than about 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, or 20 kb in length. In some embodiments, the present invention may be used to formulate and encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

The present invention may be used to formulate and encapsulate mRNA that is unmodified or mRNA containing one or more modifications that typically enhance stability. In some embodiments, modifications are selected from modified nucleotides, modified sugar phosphate backbones, and 5′ and/or 3′ untranslated region.

In some embodiments, modifications of mRNA may include modifications of the nucleotides of the RNA. A modified mRNA according to the invention can include, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine, pseudouridine, 5-methylcytidine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosure of which is included here in its full scope by reference.

Typically, mRNA synthesis includes the addition of a “cap” on the 5′ end, and a “tail” on the 3′ end. The presence of the cap is important in providing resistance to nucleases found in most eukaryotic cells. The presence of a “tail” serves to protect the mRNA from exonuclease degradation.

Thus, in some embodiments, mRNAs include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. 2′-O-methylation may also occur at the first base and/or second base following the 7-methyl guanosine triphosphate residues. Examples of cap structures include, but are not limited to, m7GpppNp-RNA, m7GpppNmp-RNA and m7GpppNmpNmp-RNA (where m indicates 2′-Omethyl residues).

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

While mRNA provided from in vitro transcription reactions may be desirable in some embodiments, other sources of mRNA are contemplated as within the scope of the invention including mRNA produced from bacteria, fungi, plants, and/or animals.

The present invention may be used to formulate and encapsulate mRNAs encoding a variety of proteins. Non-limiting examples of mRNAs suitable for the present invention include mRNAs encoding spinal motor neuron 1 (SMN), alpha-galactosidase (GLA), argininosuccinate synthetase (ASS1), ornithine transcarbamylase (OTC), Factor IX (FIX), phenylalanine hydroxylase (PAH), erythropoietin (EPO), cystic fibrosis transmembrane conductance receptor (CFTR) and firefly luciferase (FFL). Exemplary mRNA sequences as disclosed herein are listed below:

Codon-Optimized Human OTC Coding Sequence
(SEQ ID NO: 1)
AUGCUGUUCAACCUUCGGAUCUUGCUGAACAACGCUGCGUUCCGGAAUGGUCACA
ACUUCAUGGUCCGGAACUUCAGAUGCGGCCAGCCGCUCCAGAACAAGGUGCAGCU
CAAGGGGAGGGACCUCCUCACCCUGAAAAACUUCACCGGAGAAGAGAUCAAGUAC
AUGCUGUGGCUGUCAGCCGACCUCAAAUUCCGGAUCAAGCAGAAGGGCGAAUACC
UUCCUUUGCUGCAGGGAAAGUCCCUGGGGAUGAUCUUCGAGAAGCGCAGCACUCG
CACUAGACUGUCAACUGAAACCGGCUUCGCGCUGCUGGGAGGACACCCCUGCUUC
CUGACCACCCAAGAUAUCCAUCUGGGUGUGAACGAAUCCCUCACCGACACAGCGC
GGGUGCUGUCGUCCAUGGCAGACGCGGUCCUCGCCCGCGUGUACAAGCAGUCUGA
UCUGGACACUCUGGCCAAGGAAGCCUCCAUUCCUAUCAUUAAUGGAUUGUCCGAC
CUCUACCAUCCCAUCCAGAUUCUGGCCGAUUAUCUGACUCUGCAAGAACAUUACA
GCUCCCUGAAGGGGCUUACCCUUUCGUGGAUCGGCGACGGCAACAACAUUCUGCA
CAGCAUUAUGAUGAGCGCUGCCAAGUUUGGAAUGCACCUCCAAGCAGCGACCCCG
AAGGGAUACGAGCCAGACGCCUCCGUGACGAAGCUGGCUGAGCAGUACGCCAAGG
AGAACGGCACUAAGCUGCUGCUCACCAACGACCCUCUCGAAGCCGCCCACGGUGG
CAACGUGCUGAUCACCGAUACCUGGAUCUCCAUGGGACAGGAGGAGGAAAAGAA
GAAGCGCCUGCAAGCAUUUCAGGGGUACCAGGUGACUAUGAAAACCGCCAAGGUC
GCCGCCUCGGACUGGACCUUCUUGCACUGUCUGCCCAGAAAGCCCGAAGAGGUGG
ACGACGAGGUGUUCUACAGCCCGCGGUCGCUGGUCUUUCCGGAGGCCGAAAACAG
GAAGUGGACUAUCAUGGCCGUGAUGGUGUCCCUGCUGACCGAUUACUCCCCGCAG
CUGCAGAAACCAAAGUUCUGA
Codon-Optimized Human ASS1 Coding Sequence
(SEQ ID NO: 2)
AUGAGCAGCAAGGGCAGCGUGGUGCUGGCCUACAGCGGCGGCCUGGACACCAGCU
GCAUCCUGGUGUGGCUGAAGGAGCAGGGCUACGACGUGAUCGCCUACCUGGCCAA
CAUCGGCCAGAAGGAGGACUUCGAGGAGGCCCGCAAGAAGGCCCUGAAGCUGGGC
GCCAAGAAGGUGUUCAUCGAGGACGUGAGCCGCGAGUUCGUGGAGGAGUUCAUC
UGGCCCGCCAUCCAGAGCAGCGCCCUGUACGAGGACCGCUACCUGCUGGGCACCA
GCCUGGCCCGCCCCUGCAUCGCCCGCAAGCAGGUGGAGAUCGCCCAGCGCGAGGG
CGCCAAGUACGUGAGCCACGGCGCCACCGGCAAGGGCAACGACCAGGUGCGCUUC
GAGCUGAGCUGCUACAGCCUGGCCCCCCAGAUCAAGGUGAUCGCCCCCUGGCGCA
UGCCCGAGUUCUACAACCGCUUCAAGGGCCGCAACGACCUGAUGGAGUACGCCAA
GCAGCACGGCAUCCCCAUCCCCGUGACCCCCAAGAACCCCUGGAGCAUGGACGAG
AACCUGAUGCACAUCAGCUACGAGGCCGGCAUCCUGGAGAACCCCAAGAACCAGG
CCCCCCCCGGCCUGUACACCAAGACCCAGGACCCCGCCAAGGCCCCCAACACCCCC
GACAUCCUGGAGAUCGAGUUCAAGAAGGGCGUGCCCGUGAAGGUGACCAACGUG
AAGGACGGCACCACCCACCAGACCAGCCUGGAGCUGUUCAUGUACCUGAACGAGG
UGGCCGGCAAGCACGGCGUGGGCCGCAUCGACAUCGUGGAGAACCGCUUCAUCGG
CAUGAAGAGCCGCGGCAUCUACGAGACCCCCGCCGGCACCAUCCUGUACCACGCC
CACCUGGACAUCGAGGCCUUCACCAUGGACCGCGAGGUGCGCAAGAUCAAGCAGG
GCCUGGGCCUGAAGUUCGCCGAGCUGGUGUACACCGGCUUCUGGCACAGCCCCGA
GUGCGAGUUCGUGCGCCACUGCAUCGCCAAGAGCCAGGAGCGCGUGGAGGGCAAG
GUGCAGGUGAGCGUGCUGAAGGGCCAGGUGUACAUCCUGGGCCGCGAGAGCCCCC
UGAGCCUGUACAACGAGGAGCUGGUGAGCAUGAACGUGCAGGGCGACUACGAGC
CCACCGACGCCACCGGCUUCAUCAACAUCAACAGCCUGCGCCUGAAGGAGUACCA
CCGCCUGCAGAGCAAGGUGACCGCCAAGUGA
Codon-Optimized Human CFTR Coding Sequence
(SEQ ID NO: 3)
AUGCAACGCUCUCCUCUUGAAAAGGCCUCGGUGGUGUCCAAGCUCUUCUUCUCGU
GGACUAGACCCAUCCUGAGAAAGGGGUACAGACAGCGCUUGGAGCUGUCCGAUA
UCUAUCAAAUCCCUUCCGUGGACUCCGCGGACAACCUGUCCGAGAAGCUCGAGAG
AGAAUGGGACAGAGAACUCGCCUCAAAGAAGAACCCGAAGCUGAUUAAUGCGCU
UAGGCGGUGCUUUUUCUGGCGGUUCAUGUUCUACGGCAUCUUCCUCUACCUGGGA
GAGGUCACCAAGGCCGUGCAGCCCCUGUUGCUGGGACGGAUUAUUGCCUCCUACG
ACCCCGACAACAAGGAAGAAAGAAGCAUCGCUAUCUACUUGGGCAUCGGUCUGUG
CCUGCUUUUCAUCGUCCGGACCCUCUUGUUGCAUCCUGCUAUUUUCGGCCUGCAU
CACAUUGGCAUGCAGAUGAGAAUUGCCAUGUUUUCCCUGAUCUACAAGAAAACU
CUGAAGCUCUCGAGCCGCGUGCUUGACAAGAUUUCCAUCGGCCAGCUCGUGUCCC
UGCUCUCCAACAAUCUGAACAAGUUCGACGAGGGCCUCGCCCUGGCCCACUUCGU
GUGGAUCGCCCCUCUGCAAGUGGCGCUUCUGAUGGGCCUGAUCUGGGAGCUGCUG
CAAGCCUCGGCAUUCUGUGGGCUGGGAUUCCUGAUCGUGCUGGCACUGUUCCAGG
CCGGACUGGGGCGGAUGAUGAUGAAGUACAGGGACCAGAGAGCCGGAAAGAUUU
CCGAACGGCUGGUGAUCACUUCGGAAAUGAUCGAAAACAUCCAGUCAGUGAAGG
CCUACUGCUGGGAAGAGGCCAUGGAAAAGAUGAUUGAAAACCUCCGGCAAACCG
AGCUGAAGCUGACCCGCAAGGCCGCUUACGUGCGCUAUUUCAACUCGUCCGCUUU
CUUCUUCUCCGGGUUCUUCGUGGUGUUUCUCUCCGUGCUCCCCUACGCCCUGAUU
AAGGGAAUCAUCCUCAGGAAGAUCUUCACCACCAUUUCCUUCUGUAUCGUGCUCC
GCAUGGCCGUGACCCGGCAGUUCCCAUGGGCCGUGCAGACUUGGUACGACUCCCU
GGGAGCCAUUAACAAGAUCCAGGACUUCCUUCAAAAGCAGGAGUACAAGACCCUC
GAGUACAACCUGACUACUACCGAGGUCGUGAUGGAAAACGUCACCGCCUUUUGGG
AGGAGGGAUUUGGCGAACUGUUCGAGAAGGCCAAGCAGAACAACAACAACCGCA
AGACCUCGAACGGUGACGACUCCCUCUUCUUUUCAAACUUCAGCCUGCUCGGGAC
GCCCGUGCUGAAGGACAUUAACUUCAAGAUCGAAAGAGGACAGCUCCUGGCGGU
GGCCGGAUCGACCGGAGCCGGAAAGACUUCCCUGCUGAUGGUGAUCAUGGGAGA
GCUUGAACCUAGCGAGGGAAAGAUCAAGCACUCCGGCCGCAUCAGCUUCUGUAGC
CAGUUUUCCUGGAUCAUGCCCGGAACCAUUAAGGAAAACAUCAUCUUCGGCGUGU
CCUACGAUGAAUACCGCUACCGGUCCGUGAUCAAAGCCUGCCAGCUGGAAGAGGA
UAUUUCAAAGUUCGCGGAGAAAGAUAACAUCGUGCUGGGCGAAGGGGGUAUUAC
CUUGUCGGGGGGCCAGCGGGCUAGAAUCUCGCUGGCCAGAGCCGUGUAUAAGGAC
GCCGACCUGUAUCUCCUGGACUCCCCCUUCGGAUACCUGGACGUCCUGACCGAAA
AGGAGAUCUUCGAAUCGUGCGUGUGCAAGCUGAUGGCUAACAAGACUCGCAUCC
UCGUGACCUCCAAAAUGGAGCACCUGAAGAAGGCAGACAAGAUUCUGAUUCUGC
AUGAGGGGUCCUCCUACUUUUACGGCACCUUCUCGGAGUUGCAGAACUUGCAGCC
CGACUUCUCAUCGAAGCUGAUGGGUUGCGACAGCUUCGACCAGUUCUCCGCCGAA
AGAAGGAACUCGAUCCUGACGGAAACCUUGCACCGCUUCUCUUUGGAAGGCGACG
CCCCUGUGUCAUGGACCGAGACUAAGAAGCAGAGCUUCAAGCAGACCGGGGAAUU
CGGCGAAAAGAGGAAGAACAGCAUCUUGAACCCCAUUAACUCCAUCCGCAAGUUC
UCAAUCGUGCAAAAGACGCCACUGCAGAUGAACGGCAUUGAGGAGGACUCCGACG
AACCCCUUGAGAGGCGCCUGUCCCUGGUGCCGGACAGCGAGCAGGGAGAAGCCAU
CCUGCCUCGGAUUUCCGUGAUCUCCACUGGUCCGACGCUCCAAGCCCGGCGGCGG
CAGUCCGUGCUGAACCUGAUGACCCACAGCGUGAACCAGGGCCAAAACAUUCACC
GCAAGACUACCGCAUCCACCCGGAAAGUGUCCCUGGCACCUCAAGCGAAUCUUAC
CGAGCUCGACAUCUACUCCCGGAGACUGUCGCAGGAAACCGGGCUCGAAAUUUCC
GAAGAAAUCAACGAGGAGGAUCUGAAAGAGUGCUUCUUCGACGAUAUGGAGUCG
AUACCCGCCGUGACGACUUGGAACACUUAUCUGCGGUACAUCACUGUGCACAAGU
CAUUGAUCUUCGUGCUGAUUUGGUGCCUGGUGAUUUUCCUGGCCGAGGUCGCGG
CCUCACUGGUGGUGCUCUGGCUGUUGGGAAACACGCCUCUGCAAGACAAGGGAAA
CUCCACGCACUCGAGAAACAACAGCUAUGCCGUGAUUAUCACUUCCACCUCCUCU
UAUUACGUGUUCUACAUCUACGUCGGAGUGGCGGAUACCCUGCUCGCGAUGGGU
UUCUUCAGAGGACUGCCGCUGGUCCACACCUUGAUCACCGUCAGCAAGAUUCUGC
ACCACAAGAUGUUGCAUAGCGUGCUGCAGGCCCCCAUGUCCACCCUCAACACUCU
GAAGGCCGGAGGCAUUCUGAACAGAUUCUCCAAGGACAUCGCUAUCCUGGACGAU
CUCCUGCCGCUUACCAUCUUUGACUUCAUCCAGCUGCUGCUGAUCGUGAUUGGAG
CAAUCGCAGUGGUGGCGGUGCUGCAGCCUUACAUUUUCGUGGCCACUGUGCCGGU
CAUUGUGGCGUUCAUCAUGCUGCGGGCCUACUUCCUCCAAACCAGCCAGCAGCUG
AAGCAACUGGAAUCCGAGGGACGAUCCCCCAUCUUCACUCACCUUGUGACGUCGU
UGAAGGGACUGUGGACCCUCCGGGCUUUCGGACGGCAGCCCUACUUCGAAACCCU
CUUCCACAAGGCCCUGAACCUCCACACCGCCAAUUGGUUCCUGUACCUGUCCACC
CUGCGGUGGUUCCAGAUGCGCAUCGAGAUGAUUUUCGUCAUCUUCUUCAUCGCGG
UCACAUUCAUCAGCAUCCUGACUACCGGAGAGGGAGAGGGACGGGUCGGAAUAA
UCCUGACCCUCGCCAUGAACAUUAUGAGCACCCUGCAGUGGGCAGUGAACAGCUC
GAUCGACGUGGACAGCCUGAUGCGAAGCGUCAGCCGCGUGUUCAAGUUCAUCGAC
AUGCCUACUGAGGGAAAACCCACUAAGUCCACUAAGCCCUACAAAAAUGGCCAGC
UGAGCAAGGUCAUGAUCAUCGAAAACUCCCACGUGAAGAAGGACGAUAUUUGGC
CCUCCGGAGGUCAAAUGACCGUGAAGGACCUGACCGCAAAGUACACCGAGGGAGG
AAACGCCAUUCUCGAAAACAUCAGCUUCUCCAUUUCGCCGGGACAGCGGGUCGGC
CUUCUCGGGCGGACCGGUUCCGGGAAGUCAACUCUGCUGUCGGCUUUCCUCCGGC
UGCUGAAUACCGAGGGGGAAAUCCAAAUUGACGGCGUGUCUUGGGAUUCCAUUA
CUCUGCAGCAGUGGCGGAAGGCCUUCGGCGUGAUCCCCCAGAAGGUGUUCAUCUU
CUCGGGUACCUUCCGGAAGAACCUGGAUCCUUACGAGCAGUGGAGCGACCAAGAA
AUCUGGAAGGUCGCCGACGAGGUCGGCCUGCGCUCCGUGAUUGAACAAUUUCCUG
GAAAGCUGGACUUCGUGCUCGUCGACGGGGGAUGUGUCCUGUCGCACGGACAUA
AGCAGCUCAUGUGCCUCGCACGGUCCGUGCUCUCCAAGGCCAAGAUUCUGCUGCU
GGACGAACCUUCGGCCCACCUGGAUCCGGUCACCUACCAGAUCAUCAGGAGGACC
CUGAAGCAGGCCUUUGCCGAUUGCACCGUGAUUCUCUGCGAGCACCGCAUCGAGG
CCAUGCUGGAGUGCCAGCAGUUCCUGGUCAUCGAGGAGAACAAGGUCCGCCAAUA
CGACUCCAUUCAAAAGCUCCUCAACGAGCGGUCGCUGUUCAGACAAGCUAUUUCA
CCGUCCGAUAGAGUGAAGCUCUUCCCGCAUCGGAACAGCUCAAAGUGCAAAUCGA
AGCCGCAGAUCGCAGCCUUGAAGGAAGAGACUGAGGAAGAGGUGCAGGACACCC
GGCUUUAA
Comparison Codon-Optimized Human CFTR mRNA Coding Sequence
(SEQ ID NO: 4)
AUGCAGCGGUCCCCGCUCGAAAAGGCCAGUGUCGUGUCCAAACUCUUCUUCUCAU
GGACUCGGCCUAUCCUUAGAAAGGGGUAUCGGCAGAGGCUUGAGUUGUCUGACA
UCUACCAGAUCCCCUCGGUAGAUUCGGCGGAUAACCUCUCGGAGAAGCUCGAACG
GGAAUGGGACCGCGAACUCGCGUCUAAGAAAAACCCGAAGCUCAUCAACGCACUG
AGAAGGUGCUUCUUCUGGCGGUUCAUGUUCUACGGUAUCUUCUUGUAUCUCGGG
GAGGUCACAAAAGCAGUCCAACCCCUGUUGUUGGGUCGCAUUAUCGCCUCGUACG
ACCCCGAUAACAAAGAAGAACGGAGCAUCGCGAUCUACCUCGGGAUCGGACUGUG
UUUGCUUUUCAUCGUCAGAACACUUUUGUUGCAUCCAGCAAUCUUCGGCCUCCAU
CACAUCGGUAUGCAGAUGCGAAUCGCUAUGUUUAGCUUGAUCUACAAAAAGACA
CUGAAACUCUCGUCGCGGGUGUUGGAUAAGAUUUCCAUCGGUCAGUUGGUGUCC
CUGCUUAGUAAUAACCUCAACAAAUUCGAUGAGGGACUGGCGCUGGCACAUUUC
GUGUGGAUUGCCCCGUUGCAAGUCGCCCUUUUGAUGGGCCUUAUUUGGGAGOUG
UUGCAGGCAUCUGCCUUUUGUGGCCUGGGAUUUCUGAUUGUGUUGGCAUUGUUU
CAGGCUGGGCUUGGGCGGAUGAUGAUGAAGUAUCGCGACCAGAGAGCGGGUAAA
AUCUCGGAAAGACUCGUCAUCACUUCGGAAAUGAUCGAAAACAUCCAGUCGGUCA
AAGCCUAUUGCUGGGAAGAAGCUAUGGAGAAGAUGAUUGAAAACCUCCGCCAAA
CUGAGCUGAAACUGACCCGCAAGGCGGCGUAUGUCCGGUAUUUCAAUUCGUCAGC
GUUCUUCUUUUCCGGGUUCUUCGUUGUCUUUCUCUCGGUUUUGCCUUAUGCCUUG
AUUAAGGGGAUUAUCCUCCGCAAGAUUUUCACCACGAUUUCGUUCUGCAUUGUA
UUGCGCAUGGCAGUGACACGGCAAUUUCCGUGGGCCGUGCAGACAUGGUAUGAC
UCGCUUGGAGCGAUCAACAAAAUCCAAGACUUCUUGCAAAAGCAAGAGUACAAG
ACCCUGGAGUACAAUCUUACUACUACGGAGGUAGUAAUGGAGAAUGUGACGGCU
UUUUGGGAAGAGGGUUUUGGAGAACUGUUUGAGAAAGCAAAGCAGAAUAACAAC
AACCGCAAGACCUCAAAUGGGGACGAUUCCCUGUUUUUCUCGAACUUCUCCCUGC
UCGGAACACCCGUGUUGAAGGACAUCAAUUUCAAGAUUGAGAGGGGACAGCUUC
UCGCGGUAGCGGGAAGCACUGGUGCGGGAAAAACUAGCCUCUUGAUGGUGAUUA
UGGGGGAGCUUGAGCCCAGCGAGGGGAAGAUUAAACACUCCGGGCGUAUCUCAU
UCUGUAGCCAGUUUUCAUGGAUCAUGCCCGGAACCAUUAAAGAGAACAUCAUUU
UCGGAGUAUCCUAUGAUGAGUACCGAUACAGAUCGGUCAUUAAGGCGUGCCAGU
UGGAAGAGGACAUUUCUAAGUUCGCCGAGAAGGAUAACAUCGUCUUGGGAGAAG
GGGGUAUUACAUUGUCGGGAGGGCAGCGAGCGCGGAUCAGCCUCGCGAGAGCGG
UAUACAAAGAUGCAGAUUUGUAUCUGCUUGAUUCACCGUUUGGAUACCUCGACG
UAUUGACAGAAAAAGAAAUCUUCGAGUCGUGCGUGUGUAAACUUAUGGCUAAUA
AGACGAGAAUCCUGGUGACAUCAAAAAUGGAACACCUUAAGAAGGCGGACAAGA
UCCUGAUCCUCCACGAAGGAUCGUCCUACUUUUACGGCACUUUCUCAGAGUUGCA
AAACUUGCAGCCGGACUUCUCAAGCAAACUCAUGGGGUGUGACUCAUUCGACCAG
UUCAGCGCGGAACGGCGGAACUCGAUCUUGACGGAAACGCUGCACCGAUUCUCGC
UUGAGGGUGAUGCCCCGGUAUCGUGGACCGAGACAAAGAAGCAGUCGUUUAAGC
AGACAGGAGAAUUUGGUGAGAAAAGAAAGAACAGUAUCUUGAAUCCUAUUAACU
CAAUUCGCAAGUUCUCAAUCGUCCAGAAAACUCCACUGCAGAUGAAUGGAAUUG
AAGAGGAUUCGGACGAACCCCUGGAGCGGAGGCUUAGCCUCGUGCCGGAUUCAGA
GCAAGGGGAGGCCAUUCUUCCCCGGAUUUCGGUGAUUUCAACCGGACCUACACUU
CAGGCGAGGCGAAGGCAAUCCGUGCUCAACCUCAUGACGCAUUCGGUAAACCAGG
GGCAAAACAUUCACCGCAAAACGACGGCCUCAACGAGAAAAGUGUCACUUGCACC
CCAGGCGAAUUUGACUGAACUCGACAUCUACAGCCGUAGGCUUUCGCAAGAAACC
GGACUUGAGAUCAGCGAAGAAAUCAAUGAAGAAGAUUUGAAAGAGUGUUUCUUU
GAUGACAUGGAAUCAAUCCCAGCGGUGACAACGUGGAACACAUACUUGCGUUAC
AUCACGGUGCACAAGUCCUUGAUUUUCGUCCUCAUCUGGUGUCUCGUGAUCUUUC
UCGCUGAGGUCGCAGCGUCACUUGUGGUCCUCUGGCUGCUUGGUAAUACGCCCUU
GCAAGACAAAGGCAAUUCUACACACUCAAGAAACAAUUCCUAUGCCGUGAUUAUC
ACUUCUACAAGCUCGUAUUACGUGUUUUACAUCUACGUAGGAGUGGCCGACACUC
UGCUCGCGAUGGGUUUCUUCCGAGGACUCCCACUCGUUCACACGCUUAUCACUGU
CUCCAAGAUUCUCCACCAUAAGAUGCUUCAUAGCGUACUGCAGGCUCCCAUGUCC
ACCUUGAAUACGCUCAAGGCGGGAGGUAUUUUGAAUCGCUUCUCAAAAGAUAUU
GCAAUUUUGGAUGACCUUCUGCCCCUGACGAUCUUCGACUUCAUCCAGUUGUUGC
UGAUCGUGAUUGGGGCUAUUGCAGUAGUCGCUGUCCUCCAGCCUUACAUUUUUG
UCGCGACCGUUCCGGUGAUCGUGGCGUUUAUCAUGCUGCGGGCCUAUUUCUUGCA
GACGUCACAGCAGCUUAAGCAACUGGAGUCUGAAGGGAGGUCGCCUAUCUUUAC
GCAUCUUGUGACCAGUUUGAAGGGAUUGUGGACGUUGCGCGCCUUUGGCAGGCA
GCCCUACUUUGAAACACUGUUCCACAAAGCGCUGAAUCUCCAUACGGCAAAUUGG
UUUUUGUAUUUGAGUACCCUCCGAUGGUUUCAGAUGCGCAUUGAGAUGAUUUUU
GUGAUCUUCUUUAUCGCGGUGACUUUUAUCUCCAUCUUGACCACGGGAGAGGGC
GAGGGACGGGUCGGUAUUAUCCUGACACUCGCCAUGAACAUUAUGAGCACUUUG
CAGUGGGCAGUGAACAGCUCGAUUGAUGUGGAUAGCCUGAUGAGGUCCGUUUCG
AGGGUCUUUAAGUUCAUCGACAUGCCGACGGAGGGAAAGCCCACAAAAAGUACG
AAACCCUAUAAGAAUGGGCAAUUGAGUAAGGUAAUGAUCAUCGAGAACAGUCAC
GUGAAGAAGGAUGACAUCUGGCCUAGCGGGGGUCAGAUGACCGUGAAGGACCUG
ACGGCAAAAUACACCGAGGGAGGGAACGCAAUCCUUGAAAACAUCUCGUUCAGCA
UUAGCCCCGGUCAGCGUGUGGGGUUGCUCGGGAGGACCGGGUCAGGAAAAUCGA
CGUUGCUGUCGGCCUUCUUGAGACUUCUGAAUACAGAGGGUGAGAUCCAGAUCG
ACGGCGUUUCGUGGGAUAGCAUCACCUUGCAGCAGUGGCGGAAAGCGUUUGGAG
UAAUCCCCCAAAAGGUCUUUAUCUUUAGCGGAACCUUCCGAAAGAAUCUCGAUCC
UUAUGAACAGUGGUCAGAUCAAGAGAUUUGGAAAGUCGCGGACGAGGUUGGCCU
UCGGAGUGUAAUCGAGCAGUUUCCGGGAAAACUCGACUUUGUCCUUGUAGAUGG
GGGAUGCGUCCUGUCGCAUGGGCACAAGCAGCUCAUGUGCCUGGCGCGAUCCGUC
CUCUCUAAAGCGAAAAUUCUUCUCUUGGAUGAACCUUCGGCCCAUCUGGACCCGG
UAACGUAUCAGAUCAUCAGAAGGACACUUAAGCAGGCGUUUGCCGACUGCACGG
UGAUUCUCUGUGAGCAUCGUAUCGAGGCCAUGCUCGAAUGCCAGCAAUUUCUUG
UCAUCGAAGAGAAUAAGGUCCGCCAGUACGACUCCAUCCAGAAGCUGCUUAAUGA
GAGAUCAUUGUUCCGGCAGGCGAUUUCACCAUCCGAUAGGGUGAAACUUUUUCC
ACACAGAAAUUCGUCGAAGUGCAAGUCCAAACCGCAGAUCGCGGCCUUGAAAGAA
GAGACUGAAGAAGAAGUUCAAGACACGCGUCUUUAA
Codon-Optimized Human PAH Coding Sequence
(SEQ ID NO: 5)
AUGAGCACCGCCGUGCUGGAGAACCCCGGCCUGGGCCGCAAGCUGAGCGACUUCG
GCCAGGAGACCAGCUACAUCGAGGACAACUGCAACCAGAACGGCGCCAUCAGCCU
GAUCUUCAGCCUGAAGGAGGAGGUGGGCGCCCUGGCCAAGGUGCUGCGCCUGUUC
GAGGAGAACGACGUGAACCUGACCCACAUCGAGAGCCGCCCCAGCCGCCUGAAGA
AGGACGAGUACGAGUUCUUCACCCACCUGGACAAGCGCAGCCUGCCCGCCCUGAC
CAACAUCAUCAAGAUCCUGCGCCACGACAUCGGCGCCACCGUGCACGAGCUGAGC
CGCGACAAGAAGAAGGACACCGUGCCCUGGUUCCCCCGCACCAUCCAGGAGCUGG
ACCGCUUCGCCAACCAGAUCCUGAGCUACGGCGCCGAGCUGGACGCCGACCACCC
CGGGUUCAAGGACCCCGUGUACCGCGCCCGCCGCAAGCAGUUCGCCGACAUCGCC
UACAACUACCGCCACGGCCAGCCCAUCCCCCGCGUGGAGUACAUGGAGGAGGAGA
AGAAGACCUGGGGCACCGUGUUCAAGACCCUGAAGAGCCUGUACAAGACCCACGC
CUGCUACGAGUACAACCACAUCUUCCCCCUGCUGGAGAAGUACUGCGGCUUCCAC
GAGGACAACAUCCCCCAGCUGGAGGACGUGAGCCAGUUCCUGCAGACCUGCACCG
GCUUCCGCCUGCGCCCCGUGGCCGGCCUGCUGAGCAGCCGCGACUUCCUGGGCGG
CCUGGCCUUCCGCGUGUUCCACUGCACCCAGUACAUCCGCCACGGCAGCAAGCCC
AUGUACACCCCCGAGCCCGACAUCUGCCACGAGCUGCUGGGCCACGUGCCCCUGU
UCAGCGACCGCAGCUUCGCCCAGUUCAGCCAGGAGAUCGGCCUGGCCAGCCUGGG
CGCCCCCGACGAGUACAUCGAGAAGCUGGCCACCAUCUACUGGUUCACCGUGGAG
UUCGGCCUGUGCAAGCAGGGCGACAGCAUCAAGGCCUACGGCGCCGGCCUGCUGA
GCAGCUUCGGCGAGCUGCAGUACUGCCUGAGCGAGAAGCCCAAGCUGCUGCCCCU
GGAGCUGGAGAAGACCGCCAUCCAGAACUACACCGUGACCGAGUUCCAGCCCCUG
UACUACGUGGCCGAGAGCUUCAACGACGCCAAGGAGAAGGUGCGCAACUUCGCCG
CCACCAUCCCCCGCCCCUUCAGCGUGCGCUACGACCCCUACACCCAGCGCAUCGAG
GUGCUGGACAACACCCAGCAGCUGAAGAUCCUGGCCGACAGCAUCAACAGCGAGA
UCGGCAUCCUGUGCAGCGCCCUGCAGAAGAUCAAGUAA

In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ ID NO: 1, SEQ ID NO: 2, SEQ ID NO:3 or SEQ ID NO: 4. In some embodiments, an mRNA suitable for the present invention comprises a nucleotide sequence identical to SEQ ID NO: 1, SEQ ID NO: 2, SEQ ID NO:3 or SEQ ID NO: 4.

mRNA Solution

mRNA may be provided in a solution to be mixed with a lipid solution such that the mRNA may be encapsulated in lipid nanoparticles. A suitable mRNA solution may be any aqueous solution containing mRNA to be encapsulated at various concentrations. For example, a suitable mRNA solution may contain an mRNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable mRNA solution may contain an mRNA at a concentration ranging from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.09 mg/ml, 0.08 mg/ml, 0.07 mg/ml, 0.06 mg/ml, or 0.05 mg/ml.

Typically, a suitable mRNA solution may also contain a buffering agent and/or salt. Generally, buffering agents can include HEPES, ammonium sulfate, sodium bicarbonate, sodium citrate, sodium acetate, potassium phosphate and sodium phosphate. In some embodiments, suitable concentration of the buffering agent may range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM. In some embodiments, suitable concentration of the buffering agent is or greater than about 0.1 mM, 0.5 mM, 1 mM, 2 mM, 4 mM, 6 mM, 8 mM, 10 mM, 15 mM, 20 mM, 25 mM, 30 mM, 35 mM, 40 mM, 45 mM, or 50 mM.

Exemplary salts can include sodium chloride, magnesium chloride, and potassium chloride. In some embodiments, suitable concentration of salts in an mRNA solution may range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM. Salt concentration in a suitable mRNA solution is or greater than about 1 mM, 5 mM, 10 mM, 20 mM, 30 mM, 40 mM, 50 mM, 60 mM, 70 mM, 80 mM, 90 mM, or 100 mM.

In some embodiments, a suitable mRNA solution may have a pH ranging from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5. In some embodiments, a suitable mRNA solution may have a pH of or no greater than about 3.5, 4.0, 4.1, 4.2, 4.3, 4.4, 4.5, 4.6, 4.7, 4.8, 4.9, 5.0, 5.2, 5.4, 5.6, 5.8, 6.0, 6.1, 6.3, and 6.5.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, the mRNA solution is prepared by mixing an mRNA stock solution with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 mi/minute, 600-1200 ml/minute, 1200-2400 mi/minute, 2400-3600 ml/minute, 3600-4800 ml/minute, 4800-6000 mi/minute, or 60420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

### Lipid Solution

According to the present invention, a lipid solution contains a mixture of lipids suitable to form lipid nanoparticles for encapsulation of mRNA. In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, or 10 mg/ml.

Any desired lipids may be mixed at any ratios suitable for encapsulating mRNAs. In some embodiments, a suitable lipid solution contains a mixture of desired lipids including cationic lipids, helper lipids (e.g. non cationic lipids and/or cholesterol lipids) and/or PEGylated lipids. In some embodiments, a suitable lipid solution contains a mixture of desired lipids including one or more cationic lipids, one or more helper lipids (e.g. non cationic lipids and/or cholesterol lipids) and one or more PEGylated lipids.

An exemplary mixture of lipids for use with the invention is composed of four lipid components: a cationic lipid, a non-cationic lipid (e.g., DSPC, DPPC, DOPE or DEPE), a cholesterol-based lipid (e.g., cholesterol) and a PEG-modified lipid (e.g., DMG-PEG2K). In some embodiments, the molar ratio of cationic lipid(s) to non-cationic lipid(s) to cholesterol-based lipid(s) to PEG-modified lipid(s) may be between about 20-50:25-35:20-50:1-5, respectively. In some embodiments, the ratio of cationic lipid(s) to non-cationic lipid(s) to cholesterol-based lipid(s) to PEG-modified lipid(s) is approximately 20:30:48.5:1.5, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

In some embodiments, a mixture of lipids for use with the invention may comprise no more than three distinct lipid components. In some embodiments, one distinct lipid component in such a mixture is a cholesterol-based or imidazol-based cationic lipid. An exemplary mixture of lipids may be composed of three lipid components: a cationic lipid (e.g., a cholesterol-based or imidazol-based cationic lipid such as ICE, HGT4001 or HGT4002), a non-cationic lipid (e.g., DSPC, DPPC, DOPE or DEPE) and a PEG-modified lipid (e.g., DMG-PEG2K). The molar ratio of cationic lipid to non-cationic lipid to PEG-modified lipid may be between about 55-65:30-40:1-15, respectively. In some embodiments, a molar ratio of cationic lipid (e.g., a cholesterol-based or imidazol-based lipid such as ICE, HGT4001 or HGT4002) to non-cationic lipid (e.g., DSPC, DPPC, DOPE or DEPE) to PEG-modified lipid (e.g., DMG-PEG2K) of 60:35:5 is particularly suitable for use with the invention.

Cationic Lipids

As used herein, the phrase “cationic lipids” refers to any of a number of lipid species that have a net positive charge at a selected pH, such as physiological pH. Several cationic lipids have been described in the literature, many of which are commercially available. Particularly suitable cationic lipids for use in the compositions and methods of the invention include those described in international patent publications WO 2010/053572 (and particularly, C12-200 described at paragraph [00225]) and WO 2012/170930, both of which are incorporated herein by reference. In certain embodiments, cationic lipids suitable for the compositions and methods of the invention include an ionizable cationic lipid described in U.S. provisional patent application 61/617,468, filed Mar. 29, 2012 (incorporated herein by reference), such as, e.g, (15Z, 18Z)—N,N-dimethyl-6-(9Z, 12Z)-octadeca-9, 12-dien-1-yl)tetracosa-15,18-dien-1-amine (HGT5000), (15Z, 18Z)—N,N-dimethyl-6-((9Z, 12Z)-octadeca-9, 12-dien-1-yl)tetracosa-4,15,18-trien-1-amine (HGT5001), and (15Z,18Z)—N,N-dimethyl-6-((9Z, 12Z)-octadeca-9, 12-dien-1-yl)tetracosa-5, 15, 18-trien-1-amine (HGT5002).

In some embodiments, cationic lipids suitable for the compositions and methods of the invention include cationic lipids such as 3,6-bis(4-(bis((9Z,12Z)-2-hydroxyoctadeca-9,12-dien-1-yl)amino)butyl)piperazine-2,5-dione (OF-02).

In some embodiments, cationic lipids suitable for the compositions and methods of the invention include a cationic lipid described in WO 2015/184256 A2 entitled “Biodegradable lipids for delivery of nucleic acids” which is incorporated by reference herein such as 3-(4-(bis(2-hydroxydodecyl)amino)butyl)-6-(4-((2-hydroxydodecyl)(2-hydroxyundecyl)amino)butyl)-1,4-dioxane-2,5-dione (Target 23), 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione (Target 24).

In some embodiments, cationic lipids suitable for the compositions and methods of the invention include a cationic lipid described in WO 2013/063468 and in U.S. provisional application entitled “Lipid Formulations for Delivery of Messenger RNA”, both of which are incorporated by reference herein. In some embodiments, a cationic lipid comprises a compound of formula I-c1-a:

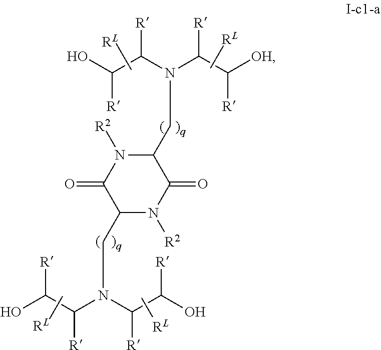

or a pharmaceutically acceptable salt thereof, wherein:
each R² independently is hydrogen or C₁₋₃ alkyl;
each q independently is 2 to 6;
each R′ independently is hydrogen or C₁₋₃ alkyl;
and each R^{L} independently is C₈₋₁₂ alkyl.

In some embodiments, each R² independently is hydrogen, methyl or ethyl. In some embodiments, each R² independently is hydrogen or methyl. In some embodiments, each R² is hydrogen.

In some embodiments, each q independently is 3 to 6. In some embodiments, each q independently is 3 to 5. In some embodiments, each q is 4.

In some embodiments, each R′ independently is hydrogen, methyl or ethyl. In some embodiments, each R′ independently is hydrogen or methyl. In some embodiments, each R′ independently is hydrogen.

In some embodiments, each R^{L} independently is C₈₋₁₂ alkyl. In some embodiments, each R^{L} independently is n-C₈₋₁₂ alkyl. In some embodiments, each R^{L} independently is C₉₋₁₁ alkyl. In some embodiments, each R^{L} independently is n-C₉₋₁₁ alkyl. In some embodiments, each R^{L} independently is C₁₀ alkyl. In some embodiments, each R^{L} independently is n-C₁₀ alkyl.

In some embodiments, each R² independently is hydrogen or methyl; each q independently is 3 to 5; each R′ independently is hydrogen or methyl; and each R^{L} independently is C₈₋₁₂ alkyl.

In some embodiments, each R² is hydrogen; each q independently is 3 to 5; each R′ is hydrogen; and each R^{L} independently is C₈₋₁₂ alkyl.

In some embodiments, each R² is hydrogen; each q is 4; each R′ is hydrogen; and each R^{L} independently is C₈₋₁₂ alkyl.

In some embodiments, a cationic lipid comprises a compound of formula I-g:

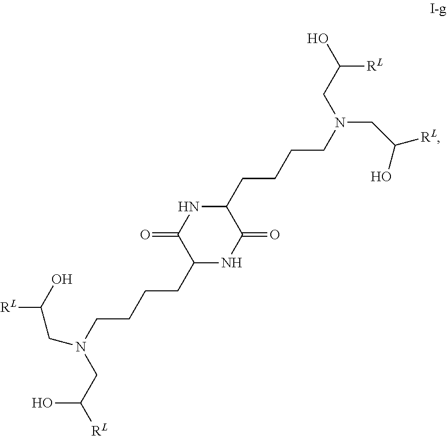

or a pharmaceutically acceptable salt thereof, wherein each R^{L} independently is C₈₋₁₂ alkyl. In some embodiments, each R^{L} independently is n-C₈₋₁₂ alkyl. In some embodiments, each R^{L} independently is C₉₋₁₁ alkyl. In some embodiments, each R^{L} independently is n-C₉₋₁₁ alkyl. In some embodiments, each R^{L} independently is C₁₀ alkyl. In some embodiments, each R^{L} is n-C₁₀ alkyl.

In particular embodiments, a suitable cationic lipid is cKK-E12, or (3,6-bis(4-(bis(2-hydroxydodecyl)amino)butyl)piperazine-2,5-dione). Structure of cKK-E12 is shown below:

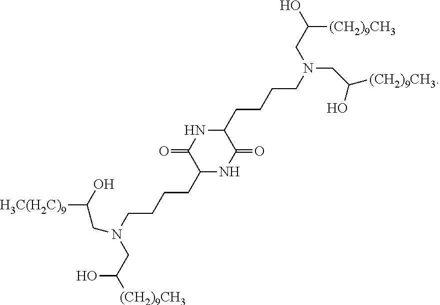

Other suitable cationic lipids include cleavable cationic lipids as described in International Patent Publication WO 2012/170889, which is incorporated herein by reference. In some embodiments, the compositions and methods of the present invention include a cationic lipid of the following formula:

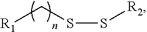

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

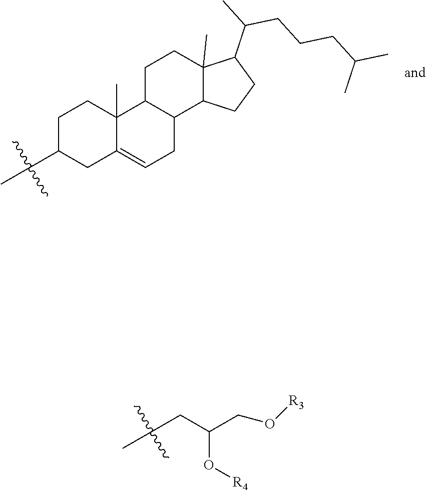

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

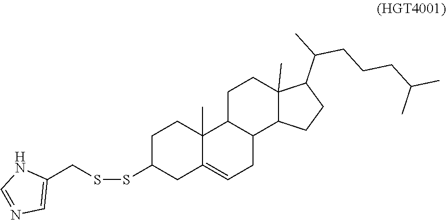 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002,” having a compound structure of:

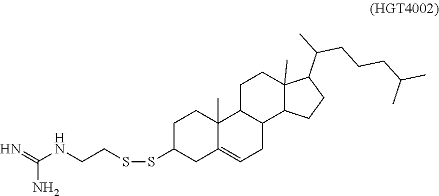 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003,” having a compound structure of:

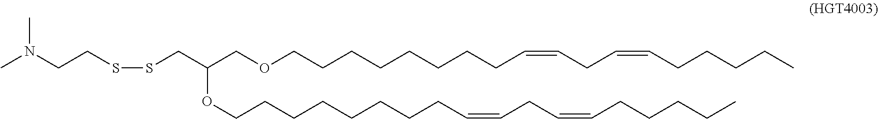 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004,” having a compound structure of:

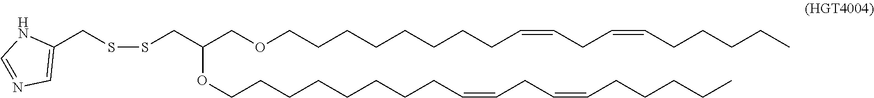 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005,” having a compound structure of:

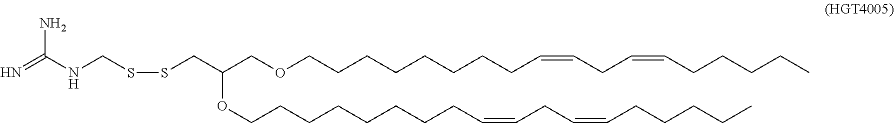 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Additional exemplary cationic lipids include those of formula I:

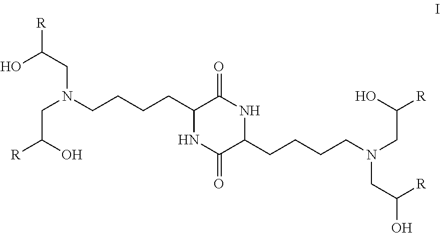

and pharmaceutically acceptable salts thereof,
wherein,

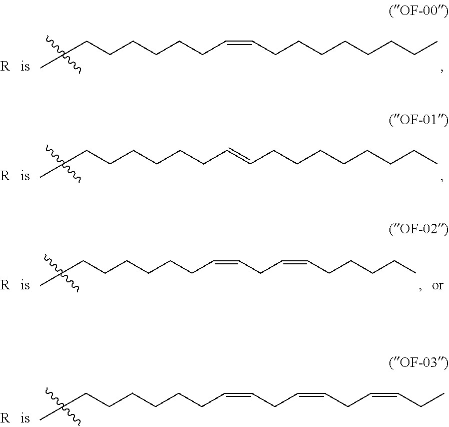

(see, e.g., Fenton, Owen S., et al. “Bioinspired Alkenyl Amino Alcohol Ionizable Lipid Materials for Highly Potent In Vivo mRNA Delivery.” Advanced materials (2016)).

In some embodiments, one or more cationic lipids suitable for the present invention may be N-[1-(2,3-dioleyloxy)propyl]-N,N,N-trimethylammonium chloride or “DOTMA”. (Feigner et al. (Proc. Nat'l Acad. Sci. 84, 7413 (1987); U.S. Pat. No. 4,897,355). Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”.

Additional exemplary cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethyl-1-(cis,cis-9′, 1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-dimethylethanamine (DLin-KC2-DMA)) (see, WO 2010/042877; Semple et al., Nature Biotech. 28: 172-176 (2010)), or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1). In some embodiments, one or more of the cationic lipids comprise at least one of an imidazole, dialkylamino, or guanidinium moiety.

In some embodiments, one or more cationic lipids may be chosen from XTC (2,2-Dilinoleyl-4-dimethylaminoethyl-[1,3]-dioxolane), MC3 (((6Z,9Z,28Z,31Z)-heptatriaconta-6,9,28,31-tetraen-19-yl 4-(dimethylamino)butanoate), ALNY-100 ((3aR,5s,6aS)-N,N-dimethyl-2,2-di((9Z,12Z)-octadeca-9,12-dienyl)tetrahydro-3aH-cyclopenta[d] [1,3]dioxol-5-amine)), NC98-5 (4,7,13-tris(3-oxo-3-(undecylamino)propyl)-N1,N16-diundecyl-4,7,10,13-tetraazahexadecane-1,16-diamide), DODAP (1,2-dioleyl-3-dimethylammonium propane), HGT4003 (WO 2012/170889, the teachings of which are incorporated herein by reference in their entirety), ICE (WO 2011/068810, the teachings of which are incorporated herein by reference in their entirety), HGT5000 (U.S. Provisional Patent Application No. 61/617,468, the teachings of which are incorporated herein by reference in their entirety) or HGT5001 (cis or trans) (Provisional Patent Application No. 61/617,468), aminoalcohol lipidoids such as those disclosed in WO2010/053572, DOTAP (1,2-dioleyl-3-trimethylammonium propane), DOTMA (1,2-di-O-octadecenyl-3-trimethylammonium propane), DLinDMA (Heyes, J.; Palmer, L.; Bremner, K.; MacLachlan, I. “Cationic lipid saturation influences intracellular delivery of encapsulated nucleic acids” J. Contr. Rel. 2005, 107, 276-287), DLin-KC2-DMA (Semple, S. C. et al. “Rational Design of Cationic Lipids for siRNA Delivery” Nature Biotech. 2010, 28, 172-176), C12-200 (Love, K. T. et al. “Lipid-like materials for low-dose in vivo gene silencing” PNAS 2010, 107, 1864-1869), N1GL, N2GL, V1GL and combinations thereof.

In some embodiments, the one or more cationic lipids are amino lipids. Amino lipids suitable for use in the invention include those described in WO2017180917, which is hereby incorporated by reference. Exemplary aminolipids in WO2017180917 include those described at paragraph [0744] such as DLin-MC3-DMA (MC3), (13Z,16Z)—N,N-dimethyl-3-nonyldocosa-13,16-dien-1-amine (L608), and Compound 18. Other amino lipids include Compound 2, Compound 23, Compound 27, Compound 10, and Compound 20. Further amino lipids suitable for use in the invention include those described in WO2017112865, which is hereby incorporated by reference. Exemplary amino lipids in WO2017112865 include a compound according to one of formulae (I), (Ia1)-(Ia6), (1b), (II), (I1a), (III), (I1ia), (IV), (17-1), (19-1), (19-11), and (20-1), and compounds of paragraphs [00185], [00201], [0276]. In some embodiments, cationic lipids suitable for use in the invention include those described in WO2016118725, which is hereby incorporated by reference. Exemplary cationic lipids in WO2016118725 include those such as KL22 and KL25. In some embodiments, cationic lipids suitable for use in the invention include those described in WO2016118724, which is hereby incorporated by reference. Exemplary cationic lipids in WO2016118725 include those such as KL10, 1,2-dilinoleyloxy-N,N-dimethylaminopropane (DLin-DMA), and KL25.

In some embodiments, cationic lipids constitute at least about 5%, 10%, 20%, 30%, 35%, 40%, 45%, 50%, 55%, 60%, 65%, or 70% of the total lipids in a suitable lipid solution by weight or by molar. In some embodiments, cationic lipid(s) constitute(s) about 30-70% (e.g., about 30-65%, about 30-60%, about 30-55%, about 30-50%, about 30-45%, about 30-40%, about 35-50%, about 35-45%, or about 3540%) of the total lipid mixture by weight or by molar.

Non-Cationic/Helper Lipids

As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 1,2-dierucoyl-sn-glycero-3-phosphoethanolamine (DEPE), 16-O-monomethyl PE, 16-O-dimethyl PE, 18-1-trans PE, 1-stearoyl-2-oleoyl-phosphatidyethanolamine (SOPE), or a mixture thereof. In some embodiments, a mixture of lipids for use with the invention may include DSPC as a non-cationic lipid component. In some embodiments, a mixture of lipids for use with the invention may include DPPC as a non-cationic lipid component. In some embodiments, a mixture of lipids for use with the invention may include DOPE as a non-cationic lipid component. In other embodiments, a mixture of lipids for use with the invention may include DEPE as a non-cationic lipid component.

In some embodiments, non-cationic lipids may constitute at least about 5%, 10%, 15%, 20%, 25%, 30%, 35%, 40%, 45%, 50%, 55%, 60%, 65% or 70% of the total lipids in a suitable lipid solution by weight or by molar. In some embodiments, non-cationic lipid(s) constitute(s) about 30-50% (e.g., about 30-45%, about 30-40%, about 35-50%, about 35-45%, or about 35-40%) of the total lipids in a suitable lipid solution by weight or by molar.

Cholesterol-Based Lipids

In some embodiments, a suitable lipid solution includes one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, cholesterol-based lipid(s) constitute(s) at least about 5%, 10%, 20%, 30%, 40%, 50%, 60%, or 70% of the total lipids in a suitable lipid solution by weight or by molar. In some embodiments, cholesterol-based lipid(s) constitute(s) about 30-50% (e.g., about 30-45%, about 30-40%, about 35-50%, about 35-45%, or about 35-40%) of the total lipids in a suitable lipid solution by weight or by molar.

PEGylated Lipids

In some embodiments, a suitable lipid solution includes one or more PEGylated lipids. For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention. Contemplated PEG-modified lipids include, but are not limited to, a polyethylene glycol chain of up to 2 kDa, up to 3 kDa, up to 4 kDa or up to 5 kDa in length covalently attached to a lipid with alkyl chain(s) of C₆-C₂₀ length. In some embodiments, a PEG-modified or PEGylated lipid is PEGylated cholesterol or PEG-2K. For example, a suitable lipid solution may include a PEG-modified lipid such as 1,2-dimyristoyl-rac-glycero-3-methoxypolyethylene glycol-2000 (DMG-PEG2K). In some embodiments, particularly useful exchangeable lipids are PEG-ceramides having shorter acyl chains (e.g., C₁₄ or C₁₈).

PEG-modified phospholipid and derivatized lipids may constitute at least about 5%, 10%, 20%, 30%, 40%, 50%, 60%, or 70% of the total lipids in a suitable lipid solution by weight or by molar. In some embodiments, the PEG-modified phospholipid and derivatized lipids constitute about 0% to about 20%, about 0.5% to about 20%, about 1% to about 15%, about 1.5% to about 5% of the total lipid present in the liposomal transfer vehicle. In some embodiments, one or more PEG-modified lipids constitute about 1.5%, about 2%, about 3% about 4% or about 5% of the total lipids by molar ratio. In some embodiments, PEGylated lipid(s) constitute(s) about 30-50% (e.g., about 30-45%, about 30-40%, about 35-50%, about 35-45%, or about 35-40%) of the total lipids in a suitable lipid solution by weight or by molar.

Various combinations of lipids, i.e., cationic lipids, non-cationic lipids, PEG-modified lipids and optionally cholesterol, that can used to prepare, and that are comprised in, pre-formed lipid nanoparticles are described in the literature and herein. For example, a suitable lipid solution may contain cKK-E12, DOPE, cholesterol, and DMG-PEG2K; C12-200, DOPE, cholesterol, and DMG-PEG2K; HGT5000, DOPE, cholesterol, and DMG-PEG2K; HGT5001, DOPE, cholesterol, and DMG-PEG2K; cKK-E12, DPPC, cholesterol, and DMG-PEG2K; C12-200, DPPC, cholesterol, and DMG-PEG2K; HGT5000, DPPC, chol, and DMG-PEG2K; HGT5001, DPPC, cholesterol, and DMG-PEG2K; or ICE, DOPE and DMG-PEG2K. Additional combinations of lipids are described in the art, e.g., U.S. Ser. No. 62/420,421 (filed on Nov. 10, 2016), U.S. Ser. No. 62/421,021 (filed on Nov. 11, 2016), U.S. Ser. No. 62/464,327 (filed on Feb. 27, 2017), and PCT Application entitled “Novel ICE-based Lipid Nanoparticle Formulation for Delivery of mRNA,” filed on Nov. 10, 2017, the disclosures of which are included here in their full scope by reference. The selection of cationic lipids, non-cationic lipids and/or PEG-modified lipids which comprise the lipid mixture as well as the relative molar ratio of such lipids to each other, is based upon the characteristics of the selected lipid(s) and the nature of the and the characteristics of the mRNA to be encapsulated. Additional considerations include, for example, the saturation of the alkyl chain, as well as the size, charge, pH, pKa, fusogenicity and toxicity of the selected lipid(s). Thus the molar ratios may be adjusted accordingly.

mRNA-LNP Formation

The process of forming LNPs encapsulating mRNA (mRNA-LNPs) by mixing a mRNA solution as described above with a lipid solution as described above, to yield a LNP formation solution suitable for mRNA-LNP formation has been described previously. For example, U.S. Pat. No. 9,668,980 entitled “Encapsulation of messenger RNA”, the entire disclosure of which is hereby incorporated in its entirety, provides a process of encapsulating messenger RNA (mRNA) in lipid nanoparticles by mixing an mRNA solution and a lipid solution, wherein the mRNA solution and/or the lipid solution are heated to a pre-determined temperature greater than ambient temperature prior to mixing, to form lipid nanoparticles that encapsulate mRNA. Alternatively, the mRNA solution and the lipid solution can be mixed into an LNP formation solution that provides for mRNA-LNP formation without heating any one or more of the mRNA solution, the lipid solution and the LNP formation solution.

For certain cationic lipid nanoparticle formulations of mRNA, in order to achieve enhance encapsulation of mRNA, the mRNA solution comprises a citrate buffer. In some embodiments, the citrate-buffered mRNA solution is heated, e.g., to 65 degrees Celsius. In those processes or methods, the heating is required to occur before the step of mixing the mRNA solution with the lipid solution (i.e. heating the separate components) as heating post-mixing of the mRNA solution with the lipid solution (post-formation of nanoparticles), heating of the LNP formation solution, has been found to not increase the encapsulation efficiency of the mRNA in the lipid nanoparticles. In some embodiments, one or both of the mRNA solution and the lipid solution are maintained and mixed at ambient temperature.

As used herein, the term “ambient temperature” refers to the temperature in a room, or the temperature which surrounds an object of interest without heating or cooling. In some embodiments, the ambient temperature at which one or more of the solutions is maintained is or is less than about 35° C., 30° C., 25° C., 20° C., or 16° C. In some embodiments, the ambient temperature at which one or more of the solutions is maintained ranges from about 15-35° C., about 15-30° C., about 15-25° C., about 15-20° C., about 20-35° C., about 25-35° C., about 30-35° C., about 20-30° C., about 25-30° C. or about 20-25° C. In some embodiments, the ambient temperature at which one or more of the solutions is maintained is 20-25° C.

Therefore, a pre-determined temperature greater than ambient temperature is typically greater than about 25° C. In some embodiments, a pre-determined temperature suitable for the present invention is or is greater than about 30° C., 37° C., 40° C., 45° C., 50° C., 55° C., 60° C., 65° C., or 70° C. In some embodiments, a pre-determined temperature suitable for the present invention ranges from about 25-70° C., about 30-70° C., about 35-70° C., about 40-70° C., about 45-70° C., about 50-70° C., or about 60-70° C. In particular embodiments, a pre-determined temperature suitable for the present invention is about 65° C.

In some embodiments, the mRNA solution or lipid solution, or both, may be heated to a pre-determined temperature above the ambient temperature prior to mixing. In some embodiments, the mRNA solution and the lipid solution are heated to the pre-determined temperature separately prior to the mixing. In some embodiments, the mRNA solution and the lipid solution are mixed at the ambient temperature but then heated to the pre-determined temperature after the mixing. In some embodiments, the lipid solution is heated to the pre-determined temperature and mixed with mRNA solution at ambient temperature. In some embodiments, the mRNA solution is heated to the pre-determined temperature and mixed with the lipid solution at ambient temperature.

In some embodiments, the mRNA solution is heated to the pre-determined temperature by adding an mRNA stock solution that is at ambient temperature to a heated buffer solution to achieve the desired pre-determined temperature.

In some embodiments, the lipid solution containing dissolved lipids may be heated to a pre-determined temperature above the ambient temperature prior to mixing. In some embodiments, the lipid solution containing dissolved lipids is heated to the pre-determined temperature separately prior to the mixing with the mRNA solution. In some embodiments, the lipid solution containing dissolved lipids is mixed at ambient temperature with the mRNA solution but then heated to a pre-determined temperature after the mixing. In some embodiments, the lipid solution containing dissolved lipids is heated to a pre-determined temperature and mixed with the mRNA solution at ambient temperature. In some embodiments, no heating of the mRNA solution, the lipid solution or the LNP formation solution occurs before or after the step of mixing one or more lipids in a lipid solution with one or more mRNAs in an mRNA solution to form mRNA encapsulated within the LNPs (mRNA-LNPs) in a LNP formation solution.

In some embodiments, the mRNA solution and the lipid solution are mixed using a pump. As the encapsulation procedure with such mixing can occur on a wide range of scales, different types of pumps may be used to accommodate desired scale. It is however generally desired to use a pulse-less flow pump. As used herein, a pulse-less flow pump refers to any pump that can establish a continuous flow with a stable flow rate. Types of suitable pumps may include, but are not limited to, gear pumps and centrifugal pumps. Exemplary gear pumps include, but are not limited to, Cole-Parmer or Diener gear pumps. Exemplary centrifugal pumps include, but are not limited to, those manufactured by Grainger or Cole-Parmer.

The mRNA solution and the lipid solution may be mixed at various flow rates. Typically, the mRNA solution may be mixed at a rate greater than that of the lipid solution. For example, the mRNA solution may be mixed at a rate at least 1×, 2×, 3×, 4×, 5×, 6×, 7×, 8×, 9×, 10×, 15×, or 20× greater than the rate of the lipid solution.

Suitable flow rates for mixing may be determined based on the scales. In some embodiments, an mRNA solution is mixed at a flow rate ranging from about 40-400 ml/minute, 60-500 ml/minute, 70-600 ml/minute, 80-700 ml/minute, 90-800 ml/minute, 100-900 ml/minute, 110-1000 ml/minute, 120-1100 ml/minute, 130-1200 ml/minute, 140-1300 ml/minute, 150-1400 ml/minute, 160-1500 ml/minute, 170-1600 ml/minute, 180-1700 ml/minute, 150-250 ml/minute, 250-500 ml/minute, 500-1000 ml/minute, 1000-2000 ml/minute, 2000-3000 ml/minute, 3000-4000 ml/minute, or 4000-5000 ml/minute. In some embodiments, the mRNA solution is mixed at a flow rate of about 200 ml/minute, about 500 ml/minute, about 1000 ml/minute, about 2000 ml/minute, about 3000 mi/minute, about 4000 ml/minute, or about 5000 ml/minute.

In some embodiments, the lipid solution is mixed at a flow rate ranging from about 25-75 ml/minute, 20-50 mL/minute, 25-75 ml/minute, 30-90 ml/minute, 40-100 ml/minute, 50-110 ml/minute, 75-200 m/minute, 200-350 ml/minute, 350-500 ml/minute, 500-650 ml/minute, 650-850 ml/minute, or 850-1000 ml/minute. In some embodiments, the lipid solution is mixed at a flow rate of about 50 mL/minute, about 100 mi/minute, about 150 ml/minute, about 200 ml/minute, about 250 ml/minute, about 300 mi/minute, about 350 ml/minute, about 400 ml/minute, about 450 ml/minute, about 500 ml/minute, about 550 ml/minute, about 600 ml/minute, about 650 mi/minute, about 700 ml/minute, about 750 ml/minute, about 800 ml/minute, about 850 ml/minute, about 900 ml/minute, about 950 ml/minute, or about 1000 ml/minute.

### Drug Product Formulation Solution

The present invention is based in part on the surprising discovery that following the mixture of mRNA solution and lipid solution into an LNP formation solution in which mRNA-encapsulated LNPs are formed, and the subsequent exchange of the LNP formation solution into a solution that constitutes the drug product formulation solution (e.g., 10% trehalose), the encapsulation of mRNA in the LNPs can be further enhanced by heating the drug product formulation solution that comprises the mRNA-LNPs as well as some free mRNA that was not encapsulated in the LNP formation solution.

The exchange of solution comprising mRNA-LNPs from LNP formation solution to drug product formulation solution can be achieved by any of a variety of buffer exchange techniques known in the art. For example, in some embodiments, this exchange of solution is achieved by diafiltration. In some embodiments, the step of exchanging the LNP formation solution for a drug product formulation solution to provide mRNA-LNP in a drug product formulation solution is accompanied by purification and/or concentration of mRNA-LNPs. Various methods may be used to achieve the exchange of solution together with purification of mRNA-LNPs or concentration of mRNA-LNPs in the solution. In some embodiments, the solution is exchange and the mRNA-LNPs are purified using Tangential Flow Filtration. Tangential flow filtration (TFF), also referred to as cross-flow filtration, is a type of filtration wherein the material to be filtered is passed tangentially across a filter rather than through it. In TFF, undesired permeate passes through the filter, while the desired retentate (mRNA-LNPs and free mRNA) passes along the filter and is collected downstream. It is important to note that the desired material is typically contained in the retentate in TFF, which is the opposite of what one normally encounters in traditional-dead end filtration.

Depending upon the material to be filtered, TFF is usually used for either microfiltration or ultrafiltration. Microfiltration is typically defined as instances where the filter has a pore size of between 0.05 μm and 1.0 μm, inclusive, while ultrafiltration typically involves filters with a pore size of less than 0.05 μm. Pore size also determines the nominal molecular weight limits (NMWL), also referred to as the molecular weight cut off (MWCO) for a particular filter, with microfiltration membranes typically having NMWLs of greater than 1,000 kilodaltons (kDa) and ultrafiltration filters having NMWLs of between 1 kDa and 1,000 kDa.

A principal advantage of tangential flow filtration is that non-permeable particles that may aggregate in and block the filter (sometimes referred to as “filter cake”) during traditional “dead-end” filtration, are instead carried along the surface of the filter. This advantage allows tangential flow filtration to be widely used in industrial processes requiring continuous operation since down time is significantly reduced because filters do not generally need to be removed and cleaned.

Tangential flow filtration can be used for several purposes including solution exchange, concentration and purification, among others. Concentration is a process whereby solvent is removed from a solution while solute molecules are retained. In order to effectively concentrate a sample, a membrane having a NMWL or MWCO that is substantially lower than the molecular weight of the solute molecules to be retained is used. Generally, one of skill may select a filter having a NMWL or MWCO of three to six times below the molecular weight of the target molecule(s).

Diafiltration is a fractionation process whereby small undesired particles are passed through a filter while larger desired nanoparticles are maintained in the retentate without changing the concentration of those nanoparticles in solution. Diafiltration is often used to remove salts or reaction buffers from a solution. Diafiltration may be either continuous or discontinuous. In continuous diafiltration, a diafiltration solution is added to the sample feed at the same rate that filtrate is generated. In discontinuous diafiltration, the solution is first diluted and then concentrated back to the starting concentration. Discontinuous diafiltration may be repeated until a desired concentration of nanoparticles is reached.

The composition of the drug product formulation solution may include various components found in drug product formulations. For example, in some embodiments, the drug product formulation solution can include a buffer such as, for example, PBS.

In some embodiments, the drug product formulation solution may include a buffering agent or salt. Exemplary buffering agent may include HEPES, ammonium sulfate, sodium bicarbonate, sodium citrate, sodium acetate, potassium phosphate and sodium phosphate. Exemplary salt may include sodium chloride, magnesium chloride, and potassium chloride.

In some embodiments, the drug product formulation solution is an aqueous solution comprising pharmaceutically acceptable excipients, including, but not limited to, a cryoprotectant. In some embodiments, the drug product formulation solution is an aqueous solution comprising pharmaceutically acceptable excipients, including, but not limited to, sugar, such as one or more of trehalose, sucrose, mannose, lactose, and mannitol. In some embodiments, the drug product formulation solution comprises trehalose. In some embodiments, the drug product formulation solution comprises sucrose. In some embodiments, the drug product formulation solution comprises mannose. In some embodiments, the drug product formulation solution comprises lactose. In some embodiments, the drug product formulation solution comprises mannitol.

In some embodiments, the drug product formulation solution is an aqueous solution comprising 5% to 20% weight to volume of a sugar, such as of trehalose, sucrose, mannose, lactose, and mannitol. In some embodiments, the drug product formulation solution is an aqueous solution comprising 5% to 20% weight to volume of trehalose. In some embodiments, the drug product formulation solution is an aqueous solution comprising 5% to 20% weight to volume of sucrose. In some embodiments, the drug product formulation solution is an aqueous solution comprising 5% to 20% weight to volume of mannose. In some embodiments, the drug product formulation solution is an aqueous solution comprising 5% to 20% weight to volume of lactose. In some embodiments, the drug product formulation solution is an aqueous solution comprising 5% to 20% weight to volume of mannitol.

In some embodiments, the drug product formulation solution is an aqueous solution comprising about 10% weight to volume of a sugar, such as of trehalose, sucrose, mannose, lactose, and mannitol. In some embodiments, the drug product formulation solution is an aqueous solution comprising about 10% weight to volume of trehalose. In some embodiments, the drug product formulation solution is an aqueous solution comprising about 10% weight to volume of sucrose. In some embodiments, the drug product formulation solution is an aqueous solution comprising about 10% weight to volume of mannose. In some embodiments, the drug product formulation solution is an aqueous solution comprising about 10% weight to volume of lactose. In some embodiments, the drug product formulation solution is an aqueous solution comprising about 10% weight to volume of mannitol.

In some embodiments, one or both of a non-aqueous solvent, such as ethanol, and citrate are absent from the drug product formulation solution. In some embodiments, the drug product formulation solution includes only residual citrate. In some embodiments, the drug product formulation solution includes only residual non-aqueous solvent, such as ethanol. In some embodiments, the drug product formulation solution contains less than about 10 mM (e.g., less than about 9 mM, about 8 mM, about 7 mM, about 6 mM, about 5 mM, about 4 mM, about 3 mM, about 2 mM, or about 1 mM) of citrate. In some embodiments, the drug product formulation solution contains less than about 25% (e.g., less than about 20%, about 15%, about 10%, about 5%, about 4%, about 3%, about 2%, or about 1%) of non-aqueous solvents, such as ethanol. In some embodiments, the drug product formulation solution does not require any further downstream processing (e.g., buffer exchange and/or further purification steps and/or additional excipients) prior to lyophilization. In some embodiments, the drug product formulation solution does not require any further downstream processing (e.g., buffer exchange and/or further purification steps and/or additional excipients) prior to administration to a sterile fill into a vial, syringe or other vessel. In some embodiments, the drug product formulation solution does not require any further downstream processing (e.g., buffer exchange and/or further purification steps and/or additional excipients) prior to administration to a subject.

In some embodiments, the drug product formulation solution has a pH between pH 4.5 and pH 7.5. In some embodiments, the drug product formulation solution has a pH between pH 5.0 and pH 7.0. In some embodiments, the drug product formulation solution has a pH between pH 5.5 and pH 7.0. In some embodiments, the drug product formulation solution has a pH above pH 4.5. In some embodiments, the drug product formulation solution has a pH above pH 5.0. In some embodiments, the drug product formulation solution has a pH above pH 5.5. In some embodiments, the drug product formulation solution has a pH above pH 6.0. In some embodiments, the drug product formulation solution has a pH above pH 6.5.

In some embodiments, the improved or enhanced amount of encapsulation of mRNA-LNPs in the drug product formulation solution following heating is retained after subsequent freeze-thaw of the drug product formulation solution. In some embodiments, the drug product formulation solution is 10% trehalose and can be stably frozen.

In some embodiments, mRNA-LNPs in the drug product formulation solution following heating can be stably frozen (e.g., retain enhanced encapsulation) in about 5%, about 10%, about 15%, about 20%, about 25%, about 30%, about 35%, about 40%, about 45%, or about 50% trehalose solution. In some embodiments, the drug product formulation solution does not require any downstream purification or processing and can be stably stored in frozen form.

Provided LNPs Encapsulating mRNA (mRNA-LNPs)

A process according to the present invention results in higher potency and efficacy thereby allowing for lower doses thereby shifting the therapeutic index in a positive direction. In some embodiments, the process according to the present invention results in homogeneous and small particle sizes. In some embodiments, the process according to the present invention results in homogeneous and small particle sizes of 200 nm or less. In some embodiments, the process according to the present invention results in homogeneous and small particle sizes of 150 nm or less. In some embodiments, the process according to the present invention results in homogeneous and small particle sizes as well as significantly improved encapsulation efficiency and/or mRNA recovery rate as compared to a prior art process.

Thus, the present invention provides a composition comprising purified mRNA-encapsulated nanoparticles described herein. In some embodiments, majority of mRNA-encapsulated nanoparticles in a composition, i.e., greater than about 50%, 55%, 60%, 65%, 70%, 75%, 80%, 85%, 90%, 95%, 96%, 97%, 98%, or 99% of the purified nanoparticles, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all of the purified nanoparticles have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). The exemplary process described herein routinely yields lipid nanoparticle compositions, in which the lipid nanoparticles have an average size of about 150 nm or less, e.g., between 75 nm and 150 nm, in particular between 100 nm and 150 nm.

In addition, homogeneous nanoparticles with narrow particle size range are achieved by a process of the present invention. For example, greater than about 70%, 75%, 80%, 85%, 90%, 95%, 96%, 97%, 98%, 99% of the purified nanoparticles in a composition provided by the present invention have a size ranging from about 75-200 nm (e.g., about 75-150 nm, about 75-140 nm, about 75-135 nm, about 75-130 nm, about 75-125 nm, about 75-120 nm, about 75-115 nm, about 75-110 nm, about 75-105 nm, about 75-100 nm, about 75-95 nm, about 75-90 nm, or 75-85 nm). In some embodiments, substantially all of the purified nanoparticles have a size ranging from about 75-200 nm (e.g., about 75-150 nm, about 75-140 nm, about 75-135 nm, about 75-130 nm, about 75-125 nm, about 75-120 nm, about 75-115 nm, about 75-110 nm, about 75-105 nm, about 75-100 nm, about 75-95 nm, about 75-90 nm, or 75-85 nm).

In some embodiments, the dispersity, or measure of heterogeneity in size of molecules (PDI), of nanoparticles in a composition provided by the present invention is less than about 0.23 (e.g., less than about 0.3, 0.2, 0.19, 0.18, 0.17, 0.16, 0.15, 0.14, 0.13, 0.12, 0.11, 0.10, 0.09, or 0.08). The exemplary process described herein routinely yields lipid nanoparticle compositions with a PDI of about 0.15 or less, e.g. between about 0.01 and 0.15.

In some embodiments, greater than about 75%, 80%, 85%, 90%, 95%, 96%, 97%, 98%, or 99% of the nanoparticles in a composition provided by the present invention encapsulate an mRNA within each individual particle. In some embodiments, substantially all of the nanoparticles in a composition encapsulate an mRNA within each individual particle.

In some embodiments, a LNP according to the present invention contains at least about 1 mg, 5 mg, 10 mg, 100 mg, 500 mg, or 1000 mg of encapsulated mRNA. In some embodiments, a process according to the present invention results in greater than about 60%, 65%, 70%, 75%, 80%, 85%, 90%, 95%, 96%, 97%, 98%, or 99% recovery of mRNA.

In some embodiments, a composition according to the present invention is formulated so as to administer doses to a subject. In some embodiments, a composition of mRNA-encapsulated LNPs as described herein is formulated at a dose concentration of less than 1.0 mg/kg mRNA lipid nanoparticles (e.g., 0.6 mg/kg, 0.5 mg/kg, 0.3 mg/kg, 0.016 mg/kg. 0.05 mg/kg, and 0.016 mg/kg. In some embodiments, the dose is decreased due to the unexpected finding that lower doses yield high potency and efficacy. In some embodiments, the dose is decreased by about 70%, 65%, 60%, 55%, 50%, 45% or 40%.

In some embodiments, the potency of mRNA-encapsulated LNPs produced by the present invention is from more than 100% (i.e., more than 200%, more than 300%, more than 400%, more than 500%, more than 600%, more than 700%, more than 800%, or more than 900%) to more than 1000% more potent when prepared by including step (c).

### EXAMPLES

While certain compounds, compositions and methods of the present invention have been described with specificity in accordance with certain embodiments, the following example serve only to illustrate the invention and are not intended to limit the same.

Lipid Materials

The formulations described in the following Example, unless otherwise specified, contain a multi-component lipid mixture of varying ratios employing one or more cationic lipids, helper lipids (e.g., non-cationic lipids and/or cholesterol lipids) and PEGylated lipids designed to encapsulate various nucleic acid materials, as discussed previously.

### Example 1. Enhanced Encapsulation of mRNA within Lipid Nanoparticles by Additional Step of Heating Drug Product Formulation Solution

This example illustrates an exemplary process of the present for enhanced encapsulation of mRNA within a lipid nanoparticle by applying Process A and subsequently exchanging the LNP formation solution comprising mRNA-LNPs and free mRNA with a drug product formulation solution and heating that drug product solution. As used herein, Process A refers to a conventional method of encapsulating mRNA by mixing mRNA with a mixture of lipids, e.g., without first pre-forming the lipids into lipid nanoparticles, as described in Published U.S. Patent Application Serial No. US2018/0008680, the entirety of which is incorporated by reference.

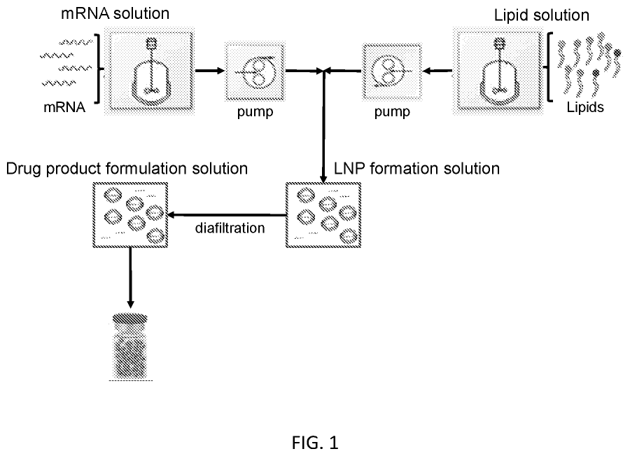

An exemplary formulation Process A is shown in FIG. 1. In this process, in some embodiments, a lipid solution in which LNP component lipids are dissolved (e.g., a solution comprising ethanol) and an aqueous mRNA solution (comprising citrate at pH 4.5) were prepared separately. In particular, the lipid solution (cationic lipid, helper lipids, zwitterionic lipids, PEG lipids etc.) was prepared by dissolving lipids in ethanol. The mRNA solution was prepared by dissolving the mRNA in citrate buffer, resulting in mRNA in citrate buffer with a pH of 4.5. The mixtures were then both heated to 65° C. prior to mixing. Then, these two solutions were mixed using a pump system to provide mRNA-encapsulated LNPs in LNP formation solution comprising a mixture of lipid solution and mRNA solution. In some instances, the two solutions were mixed using a gear pump system. In certain embodiments, the two solutions were mixing using a ‘T’ junction (or “Y” junction).

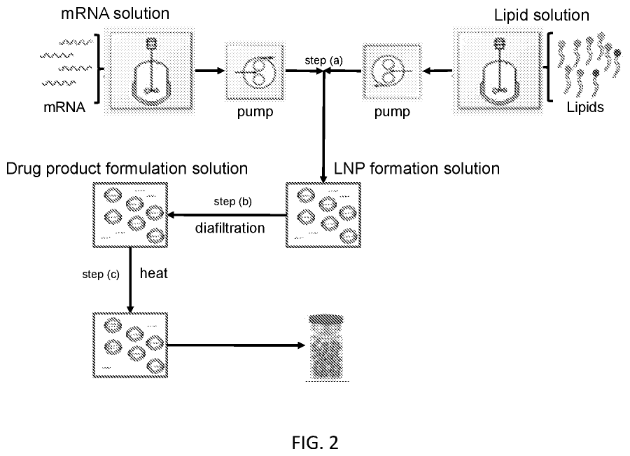

The LNP formation solution comprising mRNA-LNPs and free mRNA then was diafiltered with a TFF process. As part of that process, the LNP formation solution was removed and replaced with a drug product formulation solution comprising 10% trehalose. As shown in FIG. 2, the resultant mRNA-LNPs and free mRNA in the drug product formulation solution then was heated to 65° C. for 15 minutes. Following heating, the mRNA-LNPs and free mRNA in the drug product formulation solution was cooled and stored at 2-8° C. for subsequent analysis.

The above-described encapsulation process, as outlined in FIG. 2, was performed for 12 different mRNA-LNPs, as more specifically described in Table 1 below. For each test article, the amount of mRNA encapsulated in the formed LNPs was measured before and after heating in the drug product formulation solution of 10% trehalose, using a kit RiboGreen assay to measure free RNA according to published methods followed by a calculation to determine encapsulated mRNA. In addition, the same assay was used to measure the amount of mRNA encapsulated in the formed LNPs following subsequent freeze-thaw, to determine if the enhanced encapsulation observed from heating the mRNA-LNPs in the drug product formulation remained generally constant with subsequent freeze-thawing of the mRNA-LNPs.

TABLE 1
mRNA-LNPs prepared according to the present invention

| | | | | % | | % | Size | Size |
|---|---|---|---|---|---|---|---|---|
| | | LNP Lipid Ratio | | encapsulation | % | encapsulation | (nm)/PDI | (nm)/PDI |
| Test | Cationic | (cationic lipid:PEG- | | before | encapsulation | post | before | after |
| Article | Lipid | modified lipid:Cholesterol:DOPE) | mRNA | heating | after heating | freeze-thaw | heating | heating |
| 1 | Cationic | 40:1.5:28.5:30 | FFL | 31.6 | 78.8 | Not tested | 220.3/0.149 | 236/0.129 |
| | Lipid #1 | | | | | | | |
| 2 | Cationic | 40:3:25:32 | OTC | 69.9 | 90.6 | Not tested | 114.9/0.1 | 114.7/0.08 |
| | Lipid #2 | | | | | | | |
| 3 | Cationic | 20:1.5:48.5:30 | EPO | 75 | 80 | Not tested | 134/0.378 | 125.1/0.213 |
| | Lipid #3 | | | | | | | |
| 4 | Cationic | 20:1.5:48.5:30 | FFL | 54 | 69 | Not tested | 145.7/0.373 | 133.6/0.207 |
| | Lipid #3 | | | | | | | |
| 5 | Cationic | 20:1.5:48.5:30 | EPO | 35 | 69 | Not tested | 125.3/0.088 | 130.7/0.106 |
| | Lipid #4 | | | | | | | |
| 6 | Cationic | 20:1.5:48.5:30 | FFL | 25 | 58 | Not tested | 134.6/0.132 | 137.9/0.117 |
| | Lipid #4 | | | | | | | |
| 7 | Cationic | 40:3:25:32 | OTC | 35 | 91 | 67.7 | 120/0.20 | 118.5/0.218 |
| | Lipid #5 | | | | | | | |
| 8 | Cationic | 40:5:25:30 | OTC | 14.2 | 77.9 | 64.9 | 172.2/0.215 | 120.3/0.1 |
| | Lipid #5 | | | | | | | |
| 9 | Cationic | 40:5:25:30 | EPO | 58.5 | 73.1 | 75.3 | 116.3/0.173 | 117.3/0.15 |
| | Lipid #6 | | | | | | | |
| 10 | Cationic | 40:5:25:30 | FFL | 46.3 | 52.7 | 52.2 | 153.8/0.168 | 150.9/0.169 |
| | Lipid #6 | | | | | | | |
| 11 | Cationic | 20:1.5:48.5:30 | EPO | 29.3 | 77 | 62.8 | 161.9/0.035 | 141.2/0.024 |
| | Lipid #7 | | | | | | | |
| 12 | Cationic | 20:1.5:48.5:30 | FFL | 13.9 | 66 | 55 | 180.5/0.028 | 147.4/0.041 |
| | Lipid #7 | | | | | | | |

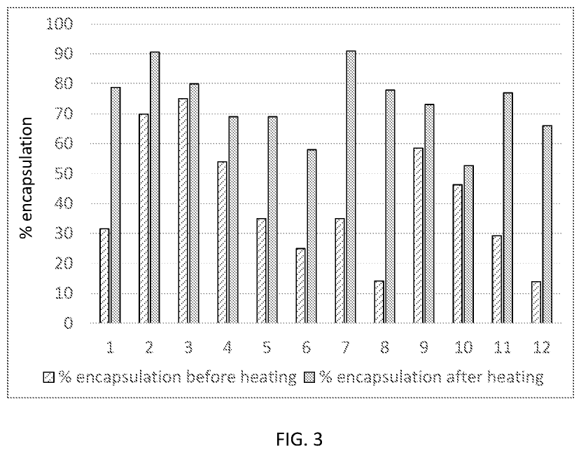

As shown in Table 1 and in FIG. 3, the % encapsulation of mRNA encapsulated in the formed LNPs was significantly following heating in the drug product formulation solution as compared to just prior to heating in the same drug product formulation solution, for all test articles assessed. Moreover, this enhanced encapsulation was maintained even following subsequent freeze-thaw of the mRNA-LNPs in the same drug product formulation solution.

Taken together, the data in this example shows that there is a substantial increase in encapsulation for mRNA-encapsulated lipid nanoparticles produced by Process A followed by heating in the drug product formulation solution.

### Example 2. In Vivo Expression of hEPO Delivered by mRNA-LNPs after Heating Drug Product Formulation Solution

This example confirms that there is a substantial increase in encapsulation for mRNA-encapsulated lipid nanoparticles produced by Process A followed by heating in the drug product formulation solution. Furthermore, the data in this example show an in vivo expression of human EPO (hEPO) in mice after administration of hEPO mRNA encapsulated in lipid nanoparticles prepared according to the present invention.

In this example, hEPO mRNA were encapsulated in lipid nanoparticles shown in Table 2, as described in Example 1. For each test article, the amount of mRNA encapsulated in the formed LNPs was measured before and after heating in the drug product formulation solution of 10 mM citrate in 10% sucrose, using a method described in example 1.

As shown in Table 2, the % encapsulation of mRNA encapsulated in the formed LNPs was significantly following heating in the drug product formulation solution as compared to just prior to heating in the same drug product formulation solution, for all test articles (each comprising different cationic lipids) assessed.

Next, mice were administered via intramuscular route, a single dose at 1 μg/30 μL of hEPO mRNA encapsulated lipid nanoparticles produced by Process A, after heating the drug formulation. Serum levels of hEPO protein were measured 6 hours and 24 hours after administration.

The levels of hEPO protein in the serum of mice after treatment can be used to evaluate the potency of mRNA via the different delivery methods. As shown in Table 2, the hEPO mRNA lipid nanoparticle formulation intramuscularly injected resulted in high levels of hEPO protein.

TABLE 2
Characteristics and in vivo expression of mRNA-LNPs prepared according
to the present invention

| | Size | | EE before | EE after | 6 hour EPO | 24 hour EPO |
|---|---|---|---|---|---|---|
| Composition | (nm) | PDI | heating | heating | (ng/mL) | (ng/mL) |
| MATE-GLA4-E16:DMG- | 117 | 0.18 | 46% | 67% | 2.89 ± 0.89 | 1.54 ± 0.33 |
| PEG:Cholesterol:DOPE | | | | | | |
| 40:1.5:28.5:30 | | | | | | |
| MATE-Suc2-E18:2:C8PEG2- | 122 | 0.48 | 50% | 73% | 5.20 ± 0.39 | 1.17 ± 0.21 |
| Ceramide:Cholesterol:DOPE | | | | | | |
| 40:1.5:28.5:30 | | | | | | |
| MATE-Suc2-E14:C8PEG2- | 119 | 0.12 | 63% | 75% | 10.33 ± 0.74 | 4.10 ± 0.27 |
| Cerimide:Cholesterol:DOPE | | | | | | |
| 40:1.5:13.5:45 | | | | | | |

### Example 3. In Vivo Expression of mRNA Delivered by Pulmonary Administration

This example confirms that there is a substantial increase in encapsulation for mRNA-encapsulated lipid nanoparticles produced by Process A followed by heating in the drug product formulation solution, which is applicable across a wide variety of cationic lipids. Furthermore, the data in this example show an in vivo expression of mRNA in mice after pulmonary administration of mRNA encapsulated in lipid nanoparticles prepared according to the present invention.

In this example, mRNA were encapsulated in lipid nanoparticles shown in Table 3, as described in Example 1. For each test article, the amount of mRNA encapsulated in the formed LNPs was measured before and after heating in the drug product formulation, using a method described in example 1.

TABLE 3
Characteristics of mRNA-LNPs prepared according to the present invention

| | | Composition | | | % EE | % EE |
|---|---|---|---|---|---|---|
| | Cationic | (DMG- | Size | | before | after |
| Sample | Lipid | PEG2000:cat:chol:DOPE) | (nm) | PDI | heating | heating |
| A | VD-3-DMA | 5:40:25:30 | 66.88 | 0.19 | 53 | 80.9 |
| B | Cationic | 5:60:0:35 | 68 | 0.127 | 57 | 92 |
| | Lipid #8 | | | | | |
| C | Cationic | 5:60:0:35 | 55 | 0.178 | 56 | 77 |
| | Lipid #9 | | | | | |
| D | Cationic | 5:40:25:30 | 72.09 | 0.13 | 29 | 93 |
| | Lipid #10 | | | | | |
| E | Cationic | 5:60:0:35 | 63 | 0.201 | 49 | 86 |
| | Lipid #11 | | | | | |
| F | TL1-10D-PIP | 3:40:25:32 | 143.2 | 0.244 | 63.8 | 76 |
| G | Cationic | 5:60:0:35 | 71.9 | 0.193 | 58 | 64 |
| | Lipid #12 | | | | | |
| H | Cationic | 5:60:0:35 | 64.8 | 0.152 | 55.0 | 89.4 |
| | Lipid #13 | | | | | |
| I | Cationic | 5:60:0:35 | 61.1 | 0.14 | 53.0 | 88.2 |
| | Lipid #14 | | | | | |
| J | Cationic | 5:60:0:35 | 55 | 0.224 | 58 | 68 |
| | Lipid #15 | | | | | |
| K | Cationic | 5:60:0:35 | 50 | 0.171 | 44 | 89 |
| | Lipid #16 | | | | | |
| L | Cationic | 5:40:25:30 | 53 | 0.204 | 59 | 89 |
| | Lipid #17 | | | | | |
| M | Cationic | 5:40:25:30 | 50 | 0.258 | 55 | 96 |
| | Lipid #18 | | | | | |

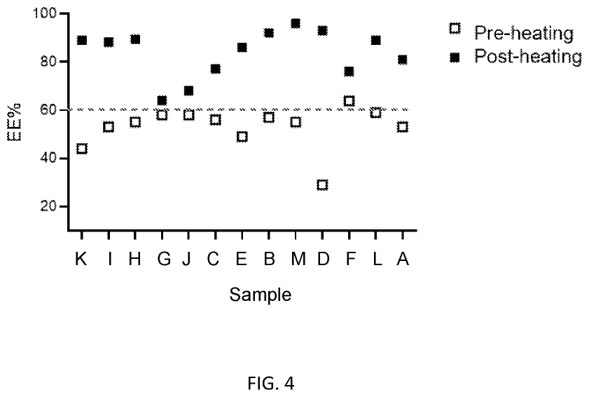

As shown in Table 3 and FIG. 4, the % encapsulation of mRNA encapsulated in the formed LNPs was significantly following heating in the drug product formulation solution as compared to just prior to heating in the same drug product formulation solution, for all test articles (each comprising different cationic lipids) assessed.

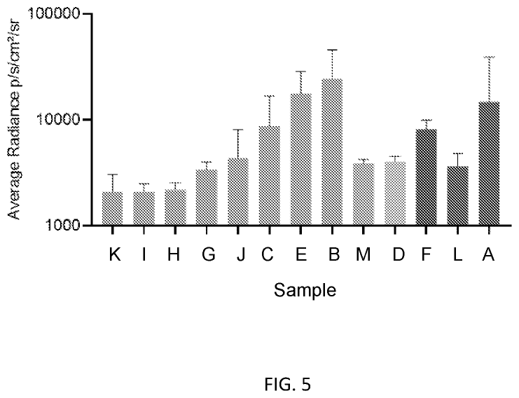

Next, mice were administered via pulmonary delivery, 10 μg of mRNA-LNPs prepared by Process A, after heating the drug formulation. Fluorescence level of the expressed protein was measured 24 hours post dosing. Protein expression as a results of the delivered mRNA was measured in p/s/cm²/sr unit, as shown in FIG. 5. The data show that mRNA lipid nanoparticle formulation administered by pulmonary delivery resulted in high levels of protein expression.

Taken together, the data in this example shows that mRNA-LNPs prepared by the present invention results in high encapsulation efficiency, which translates into high expression and potency.

### EQUIVALENTS

Those skilled in the art will recognize or be able to ascertain using no more than routine experimentation, many equivalents to the specific embodiments of the invention described herein. The scope of the present invention is not intended to be limited to the above Description, but rather is as set forth in the following claims:

We claim:

1. A process of encapsulating messenger RNA (mRNA) in lipid nanoparticles (LNPs) comprising the steps of;
(a) mixing one or more lipids in a lipid solution with one or more mRNAs in an mRNA solution to form mRNA encapsulated within the LNPs (mRNA-LNPs) in a lipid nanoparticle (LNP) formation solution;
(b) exchanging the LNP formation solution for a drug product formulation solution to provide mRNA-LNP in a drug product formulation solution; and
(c) heating the mRNA-LNP in the drug product formulation solution;
wherein the encapsulation efficiency of the mRNA-LNPs resulting from step (c) is greater than the encapsulation efficiency of the mRNA-LNPs resulting from step (b).

2. The process according to claim 1, wherein in step (a) the one or more lipids include one or more cationic lipids, one or more helper lipids, and one or more PEG-modified lipids.

3. The process according to claim 2, wherein the lipids further comprise one or more cholesterol lipids (e.g., cholesterol).

4. The process according to any one of the preceding claims, wherein in step (a) the one or more cationic lipids are selected from cKK-E12, OF-02, C12-200, MC3, DLinDMA, DLinkC2DMA, ICE (Imidazol-based), HGT5000, HGT5001, HGT4001, HGT4002, HGT4003, HGT4004, HGT4005, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, 3-(4-(bis(2-hydroxydodecyl)amino)butyl)-6-(4-((2-hydroxydodecyl)(2-hydroxyundecyl)amino)butyl)-1,4-dioxane-2,5-dione (Target 23), 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione (Target 24), N1GL, N2GL, V1GL, and combinations thereof.

5. The process according to any one of claims 2-4, wherein in step (a) the one or more helper lipids are selected from distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 1,2-dierucoyl-sn-glycero-3-phosphoethanolamine (DEPE), 16-O-monomethyl PE, 16-O-dimethyl PE, 18-1-trans PE, 1-stearoyl-2-oleoyl-phosphatidyethanolamine (SOPE), and combinations thereof.

6. The process according to claim 1, wherein in step (a) the one or more PEG-modified lipids comprise a polyethylene glycol chain of up to 2 kDa, up to 3 kDa, up to 4 kDa or up to 5 kDa in length covalently attached to a lipid with alkyl chain(s) of C₆-C₂₀ length.

7. The process according to any one of the preceding claims, wherein the lipid component of the lipid solution consists of:
(a) a cationic lipid,
(b) a helper lipid,
(c) a cholesterol-based lipid, and
(d) a PEG-modified lipid.

8. The process according to claim 8, wherein the molar ratio of the cationic lipid to helper lipid to cholesterol-based lipid to PEG-modified lipid is about 20-50:25-35:20-50:1-5.

9. The process according to any one of claims 1-6, wherein the lipid component of the lipid solution consists of:
(a) cationic lipid,
(b) a helper lipid,
(c) a PEG-modified lipid.

10. The process according to claim 9, wherein the cationic lipid is a cholesterol-based or imidazol-based cationic lipid.

11. The process according to claim 9 or 10, wherein the molar ratio of the cationic lipid to helper lipid to PEG-modified lipid is about 55-65:30-40:1-15.

12. The process according to any one of the preceding claims, wherein the mRNA encodes for a protein or peptide.

13. The process according to any one of the preceding claims, wherein in step (c) the drug product formulation solution is heated by applying heat from a heat source to the solution and the solution is maintained at a temperature greater than ambient temperature for between 10 and 20 minutes.

14. The process according to claim 13, wherein, the temperature greater than ambient temperature is about 60-70° C.

15. The process according to any one of the preceding claims, wherein the encapsulation efficiency following step (c) provides at least 5% or more over the encapsulation efficiency following step (b).

16. The process according to any one of the preceding claims, wherein the encapsulation efficiency following step (c) is improved by at least 10% or more from the encapsulation efficiency following step (b).

17. The process according to any one of the preceding claims, wherein in step (a) the lipid solution comprises lipids dissolved in ethanol.

18. The process according to any one of the preceding claims, wherein in step (a) the mRNA solution comprises mRNA dissolved in citrate buffer.

19. The process according to any one of the preceding claims, wherein the drug product formulation solution is an aqueous solution comprising pharmaceutically acceptable excipients comprising a cryoprotectant.

20. The process according to any one of the preceding claims, wherein the drug product formulation solution is an aqueous solution comprising sugar.

21. The process according to claim 20, wherein the sugar is selected from the group consisting of one or more of trehalose, sucrose, mannose, lactose, and mannitol.

22. The process according to claim 21, wherein the sugar comprises trehalose.

23. The process according to any one of the preceding claims, wherein in step (b) the drug product formulation solution is an aqueous solution comprising about 10% weight to volume of trehalose

24. The process according to any one of the preceding claims, wherein both ethanol and citrate are absent from the drug product formulation solution.

25. The process according to any one of the preceding claims, wherein the lipid solution comprises ethanol, the mRNA solution comprises citrate, and both ethanol and citrate are absent from the drug product formulation solution.

26. The process according to any one of the preceding claims, wherein the mRNA solution has a pH less than pH 5.0.

27. The process according to any one of the preceding claims, wherein the drug product formulation solution has a pH between pH 5.0 and pH 7.0.


SEQUENCE LISTING (5 sequences)

SEQ ID NO: 1 - RNA, 1065 nt, Artificial Sequence
  Synthetic polynucleotide
augcuguucaaccuucggaucuugcugaacaacgcugcguuccggaauggucacaacuucaugguccggaacuucagaugcggccagccgcuccagaacaaggugcagcucaaggggagggaccuccucacccugaaaaacuucaccggagaagagaucaaguacaugcuguggcugucagccgaccucaaauuccggaucaagcagaagggcgaauaccuuccuuugcugcagggaaagucccuggggaugaucuucgagaagcgcagcacucgcacuagacugucaacugaaaccggcuucgcgcugcugggaggacaccccugcuuccugaccacccaagauauccaucugggugugaacgaaucccucaccgacacagcgcgggugcugucguccauggcagacgcgguccucgcccgcguguacaagcagucugaucuggacacucuggccaaggaagccuccauuccuaucauuaauggauuguccgaccucuaccaucccauccagauucuggccgauuaucugacucugcaagaacauuacagcucccugaaggggcuuacccuuucguggaucggcgacggcaacaacauucugcacagcauuaugaugagcgcugccaaguuuggaaugcaccuccaagcagcgaccccgaagggauacgagccagacgccuccgugacgaagcuggcugagcaguacgccaaggagaacggcacuaagcugcugcucaccaacgacccucucgaagccgcccacgguggcaacgugcugaucaccgauaccuggaucuccaugggacaggaggaggaaaagaagaagcgccugcaagcauuucagggguaccaggugacuaugaaaaccgccaaggucgccgccucggacuggaccuucuugcacugucugcccagaaagcccgaagagguggacgacgagguguucuacagcccgcggucgcuggucuuuccggaggccgaaaacaggaaguggacuaucauggccgugauggugucccugcugaccgauuacuccccgcagcugcagaaaccaaaguucuga

SEQ ID NO: 2 - RNA, 1239 nt, Artificial Sequence
  Synthetic polynucleotide
augagcagcaagggcagcguggugcuggccuacagcggcggccuggacaccagcugcauccugguguggcugaaggagcagggcuacgacgugaucgccuaccuggccaacaucggccagaaggaggacuucgaggaggcccgcaagaaggcccugaagcugggcgccaagaagguguucaucgaggacgugagccgcgaguucguggaggaguucaucuggcccgccauccagagcagcgcccuguacgaggaccgcuaccugcugggcaccagccuggcccgccccugcaucgcccgcaagcagguggagaucgcccagcgcgagggcgccaaguacgugagccacggcgccaccggcaagggcaacgaccaggugcgcuucgagcugagcugcuacagccuggccccccagaucaaggugaucgcccccuggcgcaugcccgaguucuacaaccgcuucaagggccgcaacgaccugauggaguacgccaagcagcacggcauccccauccccgugacccccaagaaccccuggagcauggacgagaaccugaugcacaucagcuacgaggccggcauccuggagaaccccaagaaccaggccccccccggccuguacaccaagacccaggaccccgccaaggcccccaacacccccgacauccuggagaucgaguucaagaagggcgugcccgugaaggugaccaacgugaaggacggcaccacccaccagaccagccuggagcuguucauguaccugaacgagguggccggcaagcacggcgugggccgcaucgacaucguggagaaccgcuucaucggcaugaagagccgcggcaucuacgagacccccgccggcaccauccuguaccacgcccaccuggacaucgaggccuucaccauggaccgcgaggugcgcaagaucaagcagggccugggccugaaguucgccgagcugguguacaccggcuucuggcacagccccgagugcgaguucgugcgccacugcaucgccaagagccaggagcgcguggagggcaaggugcaggugagcgugcugaagggccagguguacauccugggccgcgagagcccccugagccuguacaacgaggagcuggugagcaugaacgugcagggcgacuacgagcccaccgacgccaccggcuucaucaacaucaacagccugcgccugaaggaguaccaccgccugcagagcaaggugaccgccaaguga

SEQ ID NO: 3 - RNA, 4443 nt, Artificial Sequence
  Synthetic polynucleotide
augcaacgcucuccucuugaaaaggccucggugguguccaagcucuucuucucguggacuagacccauccugagaaagggguacagacagcgcuuggagcuguccgauaucuaucaaaucccuuccguggacuccgcggacaaccuguccgagaagcucgagagagaaugggacagagaacucgccucaaagaagaacccgaagcugauuaaugcgcuuaggcggugcuuuuucuggcgguucauguucuacggcaucuuccucuaccugggagaggucaccaaggccgugcagccccuguugcugggacggauuauugccuccuacgaccccgacaacaaggaagaaagaagcaucgcuaucuacuugggcaucggucugugccugcuuuucaucguccggacccucuuguugcauccugcuauuuucggccugcaucacauuggcaugcagaugagaauugccauguuuucccugaucuacaagaaaacucugaagcucucgagccgcgugcuugacaagauuuccaucggccagcucgugucccugcucuccaacaaucugaacaaguucgacgagggccucgcccuggcccacuucguguggaucgccccucugcaaguggcgcuucugaugggccugaucugggagcugcugcaagccucggcauucugugggcuuggauuccugaucgugcuggcacuguuccaggccggacuggggcggaugaugaugaaguacagggaccagagagccggaaagauuuccgaacggcuggugaucacuucggaaaugaucgaaaacauccagucagugaaggccuacugcugggaagaggccauggaaaagaugauugaaaaccuccggcaaaccgagcugaagcugacccgcaaggccgcuuacgugcgcuauuucaacucguccgcuuucuucuucuccggguucuucgugguguuucucuccgugcuccccuacgcccugauuaagggaaucauccucaggaagaucuucaccaccauuuccuucuguaucgugcuccgcauggccgugacccggcaguucccaugggccgugcagacuugguacgacucccugggagccauuaacaagauccaggacuuccuucaaaagcaggaguacaagacccucgaguacaaccugacuacuaccgaggucgugauggaaaacgucaccgccuuuugggaggagggauuuggcgaacuguucgagaaggccaagcagaacaacaacaaccgcaagaccucgaacggugacgacucccucuucuuuucaaacuucagccugcucgggacgcccgugcugaaggacauuaacuucaagaucgaaagaggacagcuccuggcgguggccggaucgaccggagccggaaagacuucccugcugauggugaucaugggagagcuugaaccuagcgagggaaagaucaagcacuccggccgcaucagcuucuguagccaguuuuccuggaucaugcccggaaccauuaaggaaaacaucaucuucggcguguccuacgaugaauaccgcuaccgguccgugaucaaagccugccagcuggaagaggauauuucaaaguucgcggagaaagauaacaucgugcugggcgaaggggguauuaccuugucggggggccagcgggcuagaaucucgcuggccagagccguguauaaggacgccgaccuguaucuccuggacucccccuucggauaccuggacguccugaccgaaaaggagaucuucgaaucgugcgugugcaagcugauggcuaacaagacucgcauccucgugaccuccaaaauggagcaccugaagaaggcagacaagauucugauucugcaugagggguccuccuacuuuuacggcaccuucucggaguugcagaacuugcagcccgacuucucaucgaagcugauggguugcgacagcuucgaccaguucuccgccgaaagaaggaacucgauccugacggaaaccuugcaccgcuucucuuuggaaggcgacgccccugugucauggaccgagacuaagaagcagagcuucaagcagaccggggaauucggcgaaaagaggaagaacagcaucuugaaccccauuaacuccauccgcaaguucucaaucgugcaaaagacgccacugcagaugaacggcauugaggaggacuccgacgaaccccuugagaggcgccugucccuggugccggacagcgagcagggagaagccauccugccucggauuuccgugaucuccacugguccgacgcuccaagcccggcggcggcaguccgugcugaaccugaugacccacagcgugaaccagggccaaaacauucaccgcaagacuaccgcauccacccggaaagugucccuggcaccucaagcgaaucuuaccgagcucgacaucuacucccggagacugucgcaggaaaccgggcucgaaauuuccgaagaaaucaacgaggaggaucugaaagagugcuucuucgacgauauggagucgauacccgccgugacgacuuggaacacuuaucugcgguacaucacugugcacaagucauugaucuucgugcugauuuggugccuggugauuuuccuggccgaggucgcggccucacugguggugcucuggcuguugggaaacacgccucugcaagacaagggaaacuccacgcacucgagaaacaacagcuaugccgugauuaucacuuccaccuccucuuauuacguguucuacaucuacgucggaguggcggauacccugcucgcgauggguuucuucagaggacugccgcugguccacaccuugaucaccgucagcaagauucuucaccacaagauguugcauagcgugcugcaggcccccauguccacccucaacacucugaaggccggaggcauucugaacagauucuccaaggacaucgcuauccuggacgaucuccugccgcuuaccaucuuugacuucauccagcugcugcugaucgugauuggagcaaucgcagugguggcggugcugcagccuuacauuuucguggccacugugccggucauuguggcguucaucaugcugcgggccuacuuccuccaaaccagccagcagcugaagcaacuggaauccgagggacgaucccccaucuucacucaccuugugacgucguugaagggacuguggacccuccgggcuuucggacggcagcccuacuucgaaacccucuuccacaaggcccugaaccuccacaccgccaauugguuccuguaccuguccacccugcggugguuccagaugcgcaucgagaugauuuucgucaucuucuucaucgcggucacauucaucagcauccugacuaccggagagggagagggacgggucggaauaauccugacccucgccaugaacauuaugagcacccugcagugggcagugaacagcucgaucgacguggacagccugaugcgaagcgucagccgcguguucaaguucaucgacaugccuacugagggaaaacccacuaaguccacuaagcccuacaaaaauggccagcugagcaaggucaugaucaucgaaaacucccacgugaagaaggacgauauuuggcccuccggaggucaaaugaccgugaaggaccugaccgcaaaguacaccgagggaggaaacgccauucucgaaaacaucagcuucuccauuucgccgggacagcgggucggccuucucgggcggaccgguuccgggaagucaacucugcugucggcuuuccuccggcugcugaauaccgagggggaaauccaaauugacggcgugucuugggauuccauuacucugcagcaguggcggaaggccuucggcgugaucccccagaagguguucaucuucucggguaccuuccggaagaaccuggauccuuacgagcaguggagcgaccaagaaaucuggaaggucgccgacgaggucggccugcgcuccgugauugaacaauuuccuggaaagcuggacuucgugcucgucgacgggggauguguccugucgcacggacauaagcagcucaugugccucgcacgguccgugcucuccaaggccaagauucugcugcuggacgaaccuucggcccaccuggauccggucaccuaccagaucaucaggaggacccugaagcaggccuuugccgauugcaccgugauucucugcgagcaccgcaucgaggccaugcuggagugccagcaguuccuggucaucgaggagaacaagguccgccaauacgacuccauucaaaagcuccucaacgagcggucgcuguucagacaagcuauuucaccguccgauagagugaagcucuucccgcaucggaacagcucaaagugcaaaucgaagccgcagaucgcagccuugaaggaagagacugaggaagaggugcaggacacccggcuuuaa

SEQ ID NO: 4 - RNA, 4443 nt, Artificial Sequence
  Synthetic polynucleotide
augcagcgguccccgcucgaaaaggccagugucguguccaaacucuucuucucauggacucggccuauccuuagaaagggguaucggcagaggcuugaguugucugacaucuaccagauccccucgguagauucggcggauaaccucucggagaagcucgaacgggaaugggaccgcgaacucgcgucuaagaaaaacccgaagcucaucaacgcacugagaaggugcuucuucuggcgguucauguucuacgguaucuucuuguaucucggggaggucacaaaagcaguccaaccccuguuguugggucgcauuaucgccucguacgaccccgauaacaaagaagaacggagcaucgcgaucuaccucgggaucggacuguguuugcuuuucaucgucagaacacuuuuguugcauccagcaaucuucggccuccaucacaucgguaugcagaugcgaaucgcuauguuuagcuugaucuacaaaaagacacugaaacucucgucgcggguguuggauaagauuuccaucggucaguuggugucccugcuuaguaauaaccucaacaaauucgaugagggacuggcgcuggcacauuucguguggauugccccguugcaagucgcccuuuugaugggccuuauuugggagcuguugcaggcaucugccuuuuguggccugggauuucugauuguguuggcauuguuucaggcugggcuugggcggaugaugaugaaguaucgcgaccagagagcggguaaaaucucggaaagacucgucaucacuucggaaaugaucgaaaacauccagucggucaaagccuauugcugggaagaagcuauggagaagaugauugaaaaccuccgccaaacugagcugaaacugacccgcaaggcggcguauguccgguauuucaauucgucagcguucuucuuuuccggguucuucguugucuuucucucgguuuugccuuaugccuugauuaaggggauuauccuccgcaagauuuucaccacgauuucguucugcauuguauugcgcauggcagugacacggcaauuuccgugggccgugcagacaugguaugacucgcuuggagcgaucaacaaaauccaagacuucuugcaaaagcaagaguacaagacccuggaguacaaucuuacuacuacggagguaguaauggagaaugugacggcuuuuugggaagaggguuuuggagaacuguuugagaaagcaaagcagaauaacaacaaccgcaagaccucaaauggggacgauucccuguuuuucucgaacuucucccugcucggaacacccguguugaaggacaucaauuucaagauugagaggggacagcuucucgcgguagcgggaagcacuggugcgggaaaaacuagccucuugauggugauuaugggggagcuugagcccagcgaggggaagauuaaacacuccgggcguaucucauucuguagccaguuuucauggaucaugcccggaaccauuaaagagaacaucauuuucggaguauccuaugaugaguaccgauacagaucggucauuaaggcgugccaguuggaagaggacauuucuaaguucgccgagaaggauaacaucgucuugggagaaggggguauuacauugucgggagggcagcgagcgcggaucagccucgcgagagcgguauacaaagaugcagauuuguaucugcuugauucaccguuuggauaccucgacguauugacagaaaaagaaaucuucgagucgugcguguguaaacuuauggcuaauaagacgagaauccuggugacaucaaaaauggaacaccuuaagaaggcggacaagauccugauccuccacgaaggaucguccuacuuuuacggcacuuucucagaguugcaaaacuugcagccggacuucucaagcaaacucauggggugugacucauucgaccaguucagcgcggaacggcggaacucgaucuugacggaaacgcugcaccgauucucgcuugagggugaugccccgguaucguggaccgagacaaagaagcagucguuuaagcagacaggagaauuuggugagaaaagaaagaacaguaucuugaauccuauuaacucaauucgcaaguucucaaucguccagaaaacuccacugcagaugaauggaauugaagaggauucggacgaaccccuggagcgcaggcuuagccucgugccggauucagagcaaggggaggccauucuuccccggauuucggugauuucaaccggaccuacacuucaggcgaggcgaaggcaauccgugcucaaccucaugacgcauucgguaaaccaggggcaaaacauucaccgcaaaacgacggccucaacgagaaaagugucacuugcaccccaggcgaauuugacugaacucgacaucuacagccguaggcuuucgcaagaaaccggacuugagaucagcgaagaaaucaaugaagaagauuugaaagaguguuucuuugaugacauggaaucaaucccagcggugacaacguggaacacauacuugcguuacaucacggugcacaaguccuugauuuucguccucaucuggugucucgugaucuuucucgcugaggucgcagcgucacuugugguccucuggcugcuugguaauacgcccuugcaagacaaaggcaauucuacacacucaagaaacaauuccuaugccgugauuaucacuucuacaagcucguauuacguguuuuacaucuacguaggaguggccgacacucugcucgcgauggguuucuuccgaggacucccacucguucacacgcuuaucacugucuccaagauucuccaccauaagaugcuucauagcguacugcaggcucccauguccaccuugaauacgcucaaggcgggagguauuuugaaucgcuucucaaaagauauugcaauuuuggaugaccuucugccccugacgaucuucgacuucauccaguuguugcugaucgugauuggggcuauugcaguagucgcuguccuccagccuuacauuuuugucgcgaccguuccggugaucguggcguuuaucaugcugcgggccuauuucuugcagacgucacagcagcuuaagcaacuggagucugaagggaggucgccuaucuuuacgcaucuugugaccaguuugaagggauuguggacguugcgcgccuuuggcaggcagcccuacuuugaaacacuguuccacaaagcgcugaaucuccauacggcaaauugguuuuuguauuugaguacccuccgaugguuucagaugcgcauugagaugauuuuugugaucuucuuuaucgcggugacuuuuaucuccaucuugaccacgggagagggcgagggacgggucgguauuauccugacacucgccaugaacauuaugagcacuuugcagugggcagugaacagcucgauugauguggauagccugaugagguccguuucgagggucuuuaaguucaucgacaugccgacggagggaaagcccacaaaaaguacgaaacccuauaagaaugggcaauugaguaagguaaugaucaucgagaacagucacgugaagaaggaugacaucuggccuagcgggggucagaugaccgugaaggaccugacggcaaaauacaccgagggagggaacgcaauccuugaaaacaucucguucagcauuagccccggucagcgugugggguugcucgggaggaccgggucaggaaaaucgacguugcugucggccuucuugagacuucugaauacagagggugagauccagaucgacggcguuucgugggauagcaucaccuugcagcaguggcggaaagcguuuggaguaaucccccaaaaggucuuuaucuuuagcggaaccuuccgaaagaaucucgauccuuaugaacaguggucagaucaagagauuuggaaagucgcggacgagguuggccuucggaguguaaucgagcaguuuccgggaaaacucgacuuuguccuuguagaugggggaugcguccugucgcaugggcacaagcagcucaugugccuggcgcgauccguccucucuaaagcgaaaauucuucucuuggaugaaccuucggcccaucuggacccgguaacguaucagaucaucagaaggacacuuaagcaggcguuugccgacugcacggugauucucugugagcaucguaucgaggccaugcucgaaugccagcaauuucuugucaucgaagagaauaagguccgccaguacgacuccauccagaagcugcuuaaugagagaucauuguuccggcaggcgauuucaccauccgauagggugaaacuuuuuccacacagaaauucgucgaagugcaaguccaaaccgcagaucgcggccuugaaagaagagacugaagaagaaguucaagacacgcgucuuuaa

SEQ ID NO: 5 - RNA, 1359 nt, Artificial Sequence
  Synthetic polynucleotide
augagcaccgccgugcuggagaaccccggccugggccgcaagcugagcgacuucggccaggagaccagcuacaucgaggacaacugcaaccagaacggcgccaucagccugaucuucagccugaaggaggaggugggcgcccuggccaaggugcugcgccuguucgaggagaacgacgugaaccugacccacaucgagagccgccccagccgccugaagaaggacgaguacgaguucuucacccaccuggacaagcgcagccugcccgcccugaccaacaucaucaagauccugcgccacgacaucggcgccaccgugcacgagcugagccgcgacaagaagaaggacaccgugcccugguucccccgcaccauccaggagcuggaccgcuucgccaaccagauccugagcuacggcgccgagcuggacgccgaccaccccggcuucaaggaccccguguaccgcgcccgccgcaagcaguucgccgacaucgccuacaacuaccgccacggccagcccaucccccgcguggaguacauggaggaggagaagaagaccuggggcaccguguucaagacccugaagagccuguacaagacccacgccugcuacgaguacaaccacaucuucccccugcuggagaaguacugcggcuuccacgaggacaacaucccccagcuggaggacgugagccaguuccugcagaccugcaccggcuuccgccugcgccccguggccggccugcugagcagccgcgacuuccugggcggccuggccuuccgcguguuccacugcacccaguacauccgccacggcagcaagcccauguacacccccgagcccgacaucugccacgagcugcugggccacgugccccuguucagcgaccgcagcuucgcccaguucagccaggagaucggccuggccagccugggcgcccccgacgaguacaucgagaagcuggccaccaucuacugguucaccguggaguucggccugugcaagcagggcgacagcaucaaggccuacggcgccggccugcugagcagcuucggcgagcugcaguacugccugagcgagaagcccaagcugcugccccuggagcuggagaagaccgccauccagaacuacaccgugaccgaguuccagccccuguacuacguggccgagagcuucaacgacgccaaggagaaggugcgcaacuucgccgccaccaucccccgccccuucagcgugcgcuacgaccccuacacccagcgcaucgaggugcuggacaacacccagcagcugaagauccuggccgacagcaucaacagcgagaucggcauccugugcagcgcccugcagaagaucaaguaa